SEQUENCE LISTING (73 sequences)

SEQ ID NO: 1 - protein, 37 aa, Artificial Sequence
  synthetic construct
CSSVWGQCGGQNWSGPTCCASGSTCVYSNDYYSQCLP

SEQ ID NO: 2 - protein, 32 aa, Artificial Sequence
  synthetic construct
YGQCGGIGYSGPTVCASGTTCQVLNPYYSQCL

SEQ ID NO: 3 - protein, 36 aa, Artificial Sequence
  synthetic construct
TVPQWGQCGGIGYTGSTTCASPYTCHVLNPYYSQCY

SEQ ID NO: 4 - protein, 36 aa, Artificial Sequence
  synthetic construct
TQSHYGQCGGIGYSGPTVCASGTTCQYLNPYYSQCL

SEQ ID NO: 5 - protein, 36 aa, Artificial Sequence
  synthetic construct
CSSVWGQCGGQNWSGPTCCASGSTCVYSNDYYSQCL

SEQ ID NO: 6 - protein, 36 aa, Artificial Sequence
  synthetic construct
TQTHWGQCGGIGYSGCKTCTSGTTCQYSNDYYSQCL

SEQ ID NO: 7 - protein, 36 aa, Artificial Sequence
  synthetic construct
QQTVWGQCGGIGWSGPTNCAPGSACSTLNPYYAQCI

SEQ ID NO: 8 - protein, 8 aa, Artificial Sequence
  synthetic construct
NDYYSQCL

SEQ ID NO: 9 - protein, 8 aa, Artificial Sequence
  synthetic construct
NPYYSQCL

SEQ ID NO: 10 - protein, 10 aa, Artificial Sequence
  synthetic construct
GGGGYYGGGG

SEQ ID NO: 11 - protein, 11 aa, Artificial Sequence
  synthetic construct
GGGGYGYGGGG

SEQ ID NO: 12 - protein, 12 aa, Artificial Sequence
  synthetic construct
GGGGYGGYGGGG

SEQ ID NO: 13 - protein, 13 aa, Artificial Sequence
  synthetic construct
GGGGYGGGYGGGG

SEQ ID NO: 14 - protein, 11 aa, Artificial Sequence
  synthetic construct
GGGGYYYGGGG

SEQ ID NO: 15 - protein, 15 aa, Artificial Sequence
  synthetic construct
GGGGYYGGGYYGGGG

SEQ ID NO: 16 - protein, 10 aa, Artificial Sequence
  synthetic construct
GGGGWYGGGG

SEQ ID NO: 17 - protein, 14 aa, Artificial Sequence
  synthetic construct
GGGGYYSQCLGGGG

SEQ ID NO: 18 - protein, 14 aa, Artificial Sequence
  synthetic construct
GGGGYYSQCLGGGG

SEQ ID NO: 19 - protein, 10 aa, Artificial Sequence
  synthetic construct
QQQQYYQQQQ

SEQ ID NO: 20 - protein, 11 aa, Artificial Sequence
  synthetic construct
QQQQYQYQQQQ

SEQ ID NO: 21 - protein, 12 aa, Artificial Sequence
  synthetic construct
QQQQYQQYQQQQ

SEQ ID NO: 22 - protein, 13 aa, Artificial Sequence
  synthetic construct
QQQQYQQQYQQQQ

SEQ ID NO: 23 - protein, 11 aa, Artificial Sequence
  synthetic construct
QQQQYYYQQQQ

SEQ ID NO: 24 - protein, 15 aa, Artificial Sequence
  synthetic construct
QQQQYYQQQYYQQQQ

SEQ ID NO: 25 - protein, 10 aa, Artificial Sequence
  synthetic construct
QQQQWYQQQQ

SEQ ID NO: 26 - protein, 96 aa, Artificial Sequence
  synthetic construct
RCEKDERFFSDKIAKYIPIQYVLSRYPSYGLNYYQQKPVALINNQFLPYPYYAKPAAVRSSAQILQWQVLSNTVPAKSCQAQPTTMARHPHPHLSF

SEQ ID NO: 27 - protein, 12 aa, Artificial Sequence
  synthetic construct
NQQLAYFYPQLF

SEQ ID NO: 28 - protein, 12 aa, Artificial Sequence
  synthetic construct
PSGAWYYVPLGT

SEQ ID NO: 29 - protein, 4 aa, Artificial Sequence
  synthetic construct
SSSS

SEQ ID NO: 30 - protein, 4 aa, Artificial Sequence
  synthetic construct
YYYY

SEQ ID NO: 31 - protein, 4 aa, Artificial Sequence
  synthetic construct
FFFF

SEQ ID NO: 32 - protein, 4 aa, Artificial Sequence
  synthetic construct
WWWW

SEQ ID NO: 33 - protein, 4 aa, Artificial Sequence
  synthetic construct
IIII

SEQ ID NO: 34 - protein, 13 aa, Artificial Sequence
  synthetic construct
MEAESISSSEEIV

SEQ ID NO: 35 - protein, 15 aa, Artificial Sequence
  synthetic construct
ELSKAIGSESTEDQA

SEQ ID NO: 36 - protein, 110 aa, Artificial Sequence
  synthetic construct
MSLSIDVTSLPSISSSVYKNESFSTTSTISGKSIGRSEQYISPDAEAFNKYMLSKSPEDIGPSDSASNDPLTSFSIRSNAVKTNADAGVSMDSSAQSRPSSDIGFDQMDF

SEQ ID NO: 37 - protein, 70 aa, Artificial Sequence
  synthetic construct
SLNKGIKIDATMDSSISISTTSKKEKSKQENKNKYKKCYPKIEAESDSDEYVLDDSDSDDGKCKNCKYKK

SEQ ID NO: 38 - protein, 50 aa, Artificial Sequence
  synthetic construct
ITSNLVPGFIGVSSSETFLSSSSTLSTTSSRSISSSTLYENHLVNDCTNF

SEQ ID NO: 39 - protein, 80 aa, Artificial Sequence
  synthetic construct
SVGYLIGHSIVPSSSSSSSSSSSSIVVPSSRCMLLQTEKNTSIISSLCSSSTDNLNYLNSSSPHLSNHNNLHHHHYRQQQ

SEQ ID NO: 40 - protein, 21 aa, Artificial Sequence
  synthetic construct
LELSDDDDESKASINETQPPQ

SEQ ID NO: 41 - protein, 6 aa, Artificial Sequence
  synthetic construct
SYFWIP

SEQ ID NO: 42 - protein, 12 aa, Artificial Sequence
  synthetic construct
SSYYFFWWIIPP

SEQ ID NO: 43 - protein, 18 aa, Artificial Sequence
  synthetic construct
SSSYYYFFFWWWIIIPPP

SEQ ID NO: 44 - protein, 33 aa, Artificial Sequence
  synthetic construct
SSSGGGYYYGGGFFFGGGWWWGGGIIIGGGPPP

SEQ ID NO: 45 - protein, 33 aa, Artificial Sequence
  synthetic construct
SSSQQQYYYQQQFFFQQQWWWQQQIIIQQQPPP

SEQ ID NO: 46 - protein, 11 aa, Artificial Sequence
  synthetic construct
GGGGSSSGGGG

SEQ ID NO: 47 - protein, 11 aa, Artificial Sequence
  synthetic construct
QQQQSSSQQQQ

SEQ ID NO: 48 - protein, 4 aa, Artificial Sequence
  synthetic construct
AAAA

SEQ ID NO: 49 - protein, 4 aa, Artificial Sequence
  synthetic construct
YYYY

SEQ ID NO: 50 - protein, 4 aa, Artificial Sequence
  synthetic construct
MMMM

SEQ ID NO: 51 - protein, 4 aa, Artificial Sequence
  synthetic construct
VVVV

SEQ ID NO: 52 - protein, 4 aa, Artificial Sequence
  synthetic construct
LLLL

SEQ ID NO: 53 - protein, 4 aa, Artificial Sequence
  synthetic construct
WWWW

SEQ ID NO: 54 - protein, 4 aa, Artificial Sequence
  synthetic construct
IIII

SEQ ID NO: 55 - protein, 4 aa, Artificial Sequence
  synthetic construct
FFFF

SEQ ID NO: 56 - protein, 11 aa, Artificial Sequence
  synthetic construct
GGGGLLLGGGG

SEQ ID NO: 57 - protein, 11 aa, Artificial Sequence
  synthetic construct
QQQQLLLQQQQ

SEQ ID NO: 58 - protein, 11 aa, Artificial Sequence
  synthetic construct
GGGGIIIGGGG

SEQ ID NO: 59 - protein, 11 aa, Artificial Sequence
  synthetic construct
QQQQIIIQQQQ

SEQ ID NO: 60 - protein, 11 aa, Artificial Sequence
  synthetic construct
GGGGAAAGGGG

SEQ ID NO: 61 - protein, 11 aa, Artificial Sequence
  synthetic construct
QQQQAAAQQQQ

SEQ ID NO: 62 - protein, 11 aa, Artificial Sequence
  synthetic construct
GGGGVVVGGGG

SEQ ID NO: 63 - protein, 11 aa, Artificial Sequence
  synthetic construct
QQQQVVVGGGG

SEQ ID NO: 64 - protein, 11 aa, Artificial Sequence
  synthetic construct
GGGGFFFGGGG

SEQ ID NO: 65 - protein, 11 aa, Artificial Sequence
  synthetic construct
QQQQFFFQQQQ

SEQ ID NO: 66 - protein, 11 aa, Artificial Sequence
  synthetic construct
GGGGWWWGGGG

SEQ ID NO: 67 - protein, 11 aa, Artificial Sequence
  synthetic construct
QQQQWWWQQQQ

SEQ ID NO: 68 - protein, 11 aa, Artificial Sequence
  synthetic construct
GGGGYYYGGGG

SEQ ID NO: 69 - protein, 11 aa, Artificial Sequence
  synthetic construct
QQQQYYYQQQQ

SEQ ID NO: 70 - protein, 18 aa, Artificial Sequence
  synthetic construct
GGGGIIIGGGGYYYGGGG

SEQ ID NO: 71 - protein, 18 aa, Artificial Sequence
  synthetic construct
QQQQIIIQQQQYYYQQQQ

SEQ ID NO: 72 - protein, 4 aa, Artificial Sequence
  synthetic construct
KKKK

SEQ ID NO: 73 - protein, 4 aa, Artificial Sequence
  synthetic construct
RRRR

(19) United States
(12) Patent Application Publication
(10) Pub. No.: US 2014/0099444 A1
(43) Pub. Date: Apr. 10, 2014

(54) COMPOSITES CONTAINING POLYPEPTIDES ATTACHED TO POLYSACCHARIDES AND MOLECULES

(71) Applicant: The Penn State Research Foundation, University Park, PA (US)

(72) Inventors: Jeffrey M. Catchmark, Bellefonte, PA (US); Dana Meredith Mears, State College, PA (US); John Siggins, State College, PA (US); Niharika Mishra, State College, PA (US)

(73) Assignee: The Penn State Research Foundation, University Park, PA (US)

(21) Appl. No.: 14/073,833

(22) Filed: Nov. 6, 2013

Related U.S. Application Data
(63) Continuation of application No. 12/903,942, filed on Oct. 13, 2010, now abandoned.
(60) Provisional application No. 61/349,506, filed on May 28, 2010.
(60) Provisional application No. 61/250,989, filed on Oct. 13, 2009.

(51) Int. Cl. A61L 15/22; A61L 27/58; A61L 27/54; D21H 19/10; A61L 15/44; A61L 15/64; A61L 15/18; A61L 27/26; A61L 27/02

(52) U.S. Cl. CPC A61L 15/225; A61L 27/26; A61L 27/58; A61L 27/54; A61L 27/02; A61L 15/44; A61L 15/64; A61L 15/18; D21H 19/10
USPC 427/256; 427/348

(57) ABSTRACT

This document provides methods and materials related to composites or coatings containing polypeptides attached to polysaccharides and/or molecules. For example, methods and materials related to composites or coatings containing polypeptides (e.g., casein polypeptides) attached to polysaccharides (e.g., cellulose) and/or molecules (e.g., calcium containing molecules such as calcium phosphate and calcium carbonate and/or polyesters such as polylactic acid and polyhydroxybutyrate) are provided. A coating provided herein can include both cationic and ionic polymers, polypeptides, or polysaccharides.

### CROSS-REFERENCE TO RELATED APPLICATIONS

This application claims the benefit of U.S. Provisional Application Ser. No. 61/349,506, filed May 28, 2010 and U.S. Provisional Application Ser. No. 61/250,989, filed Oct. 13, 2009. The disclosures of the prior applications are considered part of (and are incorporated by reference in) the disclosure of this application.

### STATEMENT AS TO FEDERALLY SPONSORED RESEARCH

This invention was made with government support under Agreement #2007-38420-17782 and Agreement #2007-35504-18339 awarded by the United States Department of Agriculture. The government has certain rights in the invention.

### BACKGROUND

1. Technical Field

This document relates to composites or coatings containing polypeptides attached to polysaccharides and/or molecules. For example, this document provides methods and materials related to composites or coatings containing polypeptides (e.g., casein polypeptides) attached to polysaccharides (e.g., cellulose) and/or molecules (e.g., calcium containing molecules such as calcium phosphate and calcium carbonate and/or polyesters such as polylactic acid and polyhydroxybutyrate).

2. Background Information

Polysaccharides and polypeptides are common components of living organisms that can be obtained in large quantities. For example, cellulose is an abundant polysaccharide found in plant matter. Cellulose is a renewable material produced biologically in a natural process that consumes and stores carbon dioxide without the need for high temperature and high energy consuming processes. Cellulose is a major constituent of paper and cardboard, and of textiles made from cotton, linen, and other plant fibers. Casein is a polypeptide that accounts for a large percentage of the polypeptides found in milk and cheese products.

Polylactic acid (PLA) is a commercially available biologically produced polymer. It is a biodegradable thermoplastic polyester produced from L- and D-lactic acid, which can be derived from the fermentation of corn starch or sugarcanes. Other biodegradable polymers include poly-β-hydroxy butyrate-co-valerate (PHBV), polyhydroxyalkanoate (PHA), and polyhydroxybutyrade (PHB).

### SUMMARY

This document relates to composites or coatings containing polypeptides attached to (a) polysaccharides, (b) non-polypeptide, non-polysaccharide molecules, or (c) both polysaccharides and non-polypeptide, non-polysaccharide molecules. For example, this document provides methods and materials related to composites containing casein polypeptides attached to cellulose and molecules such as calcium containing molecules (e.g., calcium phosphate or calcium carbonate molecules), polyesters (e.g., polylactic acid or polyhydroxybutyrate), or other polymers such as polyethylene or polystyrene. This document also provides methods and materials for making and using such composites.

The composites provided herein can be used to produce products derived from wood. For example, the composites provided herein can be used to produce cardboard products, particleboard products, and paper products. In some cases, the composites provided herein can be used to produce a coating layer (e.g., an inner coating layer or an outer coating layer) for a cardboard product, particleboard product, or paper product. Such coatings can allow wood or paper product manufacturers to produce products having a desirable outer surface such as those found on high quality printing paper.

In some cases, the composites provided herein can be used to produce health care products and medical implants. For example, the composites provided herein can be used to produce wound care or tissue engineering products (e.g., nerve, bone, or cartilage tissue scaffolds or injectable implant materials having osteoinductive and/or bioabsorbable properties).

In general, one aspect of this document features a composite material comprising, or consisting essentially of, a polypeptide containing one or more aromatic amino acid residues and one or more phosphorylated amino acid residues, a polysaccharide attached to the polypeptide, and a non-polypeptide, non-polysaccharide molecule attached to the polypeptide. The polypeptide can comprise at least two contiguous tyrosine amino acid residues. The polypeptide can comprise at least two phosphoserine residues within 30 contiguous amino acid residues. The polypeptide can be a casein polypeptide. The polypeptide can be a κ-casein polypeptide. The polypeptide can be a αs1-casein polypeptide. The polysaccharide can be a beta 1,4 linked glucan polysaccharide. The polypeptide can be a αₛ₂-casein polypeptide. The polypeptide can be a β-casein polypeptide. The polysaccharide can be cellulose. The polysaccharide can be microbial cellulose. The polysaccharide can be starch. The polysaccharide can be chitin. The non-polypeptide, non-polysaccharide molecule can be a calcium-containing molecule. The calcium-containing molecule can be calcium phosphate or calcium carbonate. The non-polypeptide, non-polysaccharide molecule can be a polyester. The polyester can be polyhydroxybutyrate or polylactic acid. The composite can be formed at a pH greater than 7.0. The composite can be formed at a pH between 9.0 and 11.0. The composite can be made by a method comprising (a) attaching two or more of the polypeptides to calcium-containing particles in a liquid solution to form a complex, and (b) mixing the complex with a polysaccharide in an aqueous solution to form the composite. The method can comprise dehydrating the composite. The composite can comprise a casein polypeptide, cellulose, a calcium-containing molecule, and a polyester.

In another aspect, this document features a composite material comprising, or consisting essentially of, a polypeptide having two or more sequences selected from the group consisting of a sequence having one or more aromatic amino acid residues, a sequence having one or more hydrophobic amino acid residues, a sequence having one or more phosphorylated amino acid residues, and a sequence having one or more positively charged amino acid residues, a polysaccharide attached to the polypeptide, and a non-polypeptide, non-polysaccharide molecule attached to the polypeptide. The polypeptide can comprise at least two contiguous tyrosine amino acid residues. The polypeptide can comprise at least two phosphoserine residues within 30 contiguous amino acid residues. The polypeptide can be a casein polypeptide. The polypeptide can be a κ-casein polypeptide. The polypeptide can be a αs1-casein polypeptide. The polysaccharide can be a beta 1,4 linked glucan polysaccharide. The polypeptide can be a αₛ₂-casein polypeptide. The polypeptide can be a β-casein polypeptide. The polysaccharide can be cellulose. The polysaccharide can be microbial cellulose. The polysaccharide can be starch. The polysaccharide can be chitin. The non-polypeptide, non-polysaccharide molecule can be a calcium-containing molecule. The calcium-containing molecule can be calcium phosphate or calcium carbonate. The non-polypeptide, non-polysaccharide molecule can be a polyester. The polyester can be polyhydroxybutyrate or polylactic acid. The composite can be formed at a pH greater than 7.0. The composite can be formed at a pH between 9.0 and 11.0. The composite can be made by a method comprising (a) attaching two or more of the polypeptides to calcium-containing particles in a liquid solution to form a complex, and (b) mixing the complex with a polysaccharide in an aqueous solution to form the composite. The method can comprise dehydrating the composite. The composite can comprise a casein polypeptide, cellulose, a calcium-containing molecule, and a polyester.

In another aspect, this document features a polysaccharide composition microbially produced to comprise aligned polysaccharides, wherein the alignment is produced by applying an external electric or magnetic field during the culturing of microbes that produce the polysaccharides.

In another aspect, this document features a polysaccharide composition microbially produced using a template material. In this case, the microbially produced polysaccharide can be grown over or in between features of the template material to form a three dimensional structure of the polysaccharide. The template material can be degradable such that either as the polysaccharide is microbially produced or after the polysaccharide is produced, the template material can be removed. Non-microbially produced polysaccharides or polysaccharide compositions described herein also can be formed using a template. In another aspect, this document features a cellulose composition comprising nanofibers of cellulose measuring between 1 nm and 50 nm in diameter and between 50 nm and 50,000 nm in length, wherein the nanofibers are aligned such that their long axes are generally parallel, and wherein the composition comprises greater than 60% cellulose content and less than 30% free condensed water.

In another aspect, this document features a cellulose nanofiber material produced by culturing bacteria under conditions wherein the bacteria culture temperature is cycled from a temperature below the thermal stability temperature of its cellulose synthesis enzyme to a temperature above the thermal stability temperature of its cellulose synthesis enzyme.

In another aspect, this document features a composite material comprising (a) a κ-casein polypeptide and (b) chymosin or rennin. The chymosin or the rennin can be introduced into the material after the κ-casein polypeptide. The composite can comprise cellulose. The composite material can be a foam.

In another aspect, this document features a composite material comprising microbial cellulose and an olfactive component.

In another aspect, this document features a chewable, edible composite material comprising microbial cellulose and a flavoring component.

In another aspect, this document features a chewable, edible composite material comprising microbial cellulose and a nutritional component.

In another aspect, this document features a chewable, edible composite material comprising microbial cellulose and a drug compound.

In another aspect, this document features a process for forming a coated composite material, wherein the process comprises (a) applying a first coating to a substrate material to form a first coated composite substrate material using a coating liquid solution comprising a polymer, polypeptide, or polysaccharide with an opposite charge relative to the charge of the substrate material, wherein the composite substrate material comprises a cationic or anionic polymer, polypeptide, or polysaccharide, wherein the polymer, polypeptide, or polysaccharide is applied at weight loading in excess of what can effectively bind to the substrate material and (b) applying a second coating to the first coated composite substrate material using a second coating solution comprising a polymer, polypeptide, or polysaccharide with opposite charge relative to the polymer, polypeptide, or polysaccharide of the first coating.

In another aspect, this document features a formed composite substrate material comprising a cationic or anionic polymer, polypeptide, or polysaccharide, a first coating, and a second coating, wherein the first coating was applied to the substrate material using a coating liquid solution comprising a polymer, polypeptide, or polysaccharide with opposite charge relative to the substrate material, wherein the polymer, polypeptide, or polysaccharide of the first coating was applied at a weight loading in excess of what can effectively bind to the substrate material, and wherein the second coating was applied to the first coating of the substrate material using a coating solution comprising a polymer, polypeptide, or polysaccharide with opposite charge relative to the polymer, polypeptide, or polysaccharide of the first coating.

In another aspect, this document features a formed composite substrate material comprising a cationic or anionic polymer, polypeptide, or polysaccharide, a first coating, and a second coating, wherein the first coating was applied to the substrate material via a coating liquid solution comprising a polymer, polypeptide, or polysaccharide with opposite charge relative to the substrate material, and wherein the second coating was applied to the substrate material via a second coating solution comprising a mixture of two or more oppositely charged polymers, polypeptides, or polysaccharides.

In another aspect, this document features a process for forming a coated composite material, wherein the process comprises (a) applying a first coating to a substrate material via a first coating liquid solution, wherein the substrate material comprises a cationic or anionic polymer, polypeptide, or polysaccharide, wherein the first coating liquid solution comprises a polymer, polypeptide, or polysaccharide with opposite charge relative to the substrate material, wherein the polymer, polypeptide, or polysaccharide of the first coating is applied at weight loading in excess of what can effectively bind to the substrate material and (b) applying a second coating to the composite substrate material via a second coating solution comprising a polymer, polypeptide, or polysaccharide with opposite charge relative to the polymer, polypeptide, or polysaccharide of the first coating.

In another aspect, this document features a process for forming a coated composite material, wherein the process comprises applying a first coating to a substrate material via a coating liquid solution comprising a polymer, polypeptide, or polysaccharide with opposite charge relative to the substrate material, wherein the substrate material comprises a cationic or anionic polymer, polypeptide, or polysaccharide and (b) applying a second coating to the substrate material via a second coating solution comprising a mixture of two or more oppositely charged polymers, polypeptides, or polysaccharides.

In some embodiments, the substrate material can comprise cellulose. In some cases, the substrate material can consists of cellulose. The substrate material can comprise a paper composition. The substrate material can comprise a cationic polysaccharide. The cationic polysaccharide can be chitosan or cationic starch. The substrate material can comprise an anionic polysaccharide. The anionic polysaccharide can be carboxylmethylcellulose or anionic starch. The substrate material can comprise an anionic polypeptide. The anionic polypeptide can be a casein polypeptide. The total charge ratio of all cationic components and all anionic components can be approximately equal. The composite substrate material can comprise at least 50 percent cellulose.

In another aspect, this document features a engineered composite material comprising cellulose, lignin, and a polypeptide attached to the cellulose and the lignin.

In another aspect, this document features a composite material comprising a polypeptide containing one or more hydrophobic amino acid residues and a non-polypeptide, non-polysaccharide molecule attached to the polypeptide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a schematic representation of a composite containing casein polypeptides. The casein polypeptides can be attached to cellulose polysaccharides, polylactic acid (PLA), calcium phosphate, other casein polypeptides, or any combination hereof. In some cases, PLA can be replaced with other biodegradable polymers such as PHBV, PHA, or PHB.

FIG. 2A is a scanning electron micrograph of a mixture formed using cellulose and hydroxyapatite (HA) where virtually no HA is present in the compound.

FIG. 2B is a scanning electron micrograph of a composite containing cellulose, HA, and casein polypeptides where HA is clearly present in the mixture.

FIG. 3. SPR results at a fixed angle illustrating the adsorption process of casein at the gold film and PLA at the casein deposited film. Arrows indicate the times when the solutions were changed. The gold film was first flowed with running buffer, then casein solution, buffer, PLA solution and buffer.

FIG. 4. In an order of buffer/casein/buffer/PLA/buffer, SPR intensity change as a function of angle of incident light. Each solution flowed on the surface at least 10 minutes, and data were collected at the changed angles.

FIG. 5. SPR results at a fixed angle illustrating nearly no adsorption of PLA at the bare gold film. Arrows indicate the times when the solutions were changed. The gold film was first flowed with running buffer, then PLA solution and buffer.

FIG. 6. In an order of buffer/PLA/buffer, SPR intensity change as a function of angle of incident light. Each solution flowed on the surface at least 10 minutes, and data were collected at the changed angles.

FIG. 7: A schematic diagram of an exemplary holder with a feature for forming a round-like fiber bundle: (a) cross section, and (b) top view. Item 1 can be poly(lactic-co-glycolic acid); item 2 can be a shaped feature to hold and guide degradable poly(lactic-co-glycolic acid) wound around the feature; item 3 can be a base of the shaped feature; item 4 is the poly(lactic-co-glycolic acid) fibers wound around feature; item 5 is the shaped feature to hold and guide degradable poly(lactic-co-glycolic acid) wound around feature; and item 6 is the base of the feature.

FIG. 8. A schematic illustration of a culturing setup where the holder shown in FIG. 7 is submerged into a culture media growing, e.g., Acetobacter xylinum and cellulose. Item 1 shows the interface between a bacteria culture media (below line in vessel) and air (above line). The fixture (item 3) is positioned such that poly(lactic-co-glycolic acid) fibers are located below the media line (item 1). Item 2 is a vessel in which bacteria can be cultured and the feature shown in FIG. 7 can be placed. Item 3 is the feature with poly(lactic-co-glycolic acid) fibers shown in FIG. 7.

### DETAILED DESCRIPTION

This document provides methods and materials related to composites containing polypeptides attached to (a) polysaccharides, (b) non-polypeptide, non-polysaccharide molecules, or (c) both polysaccharides and non-polypeptide, non-polysaccharide molecules. This document also provides methods and materials for making and using such composites.

The composites provided herein can include one or more polypeptides (e.g., a casein polypeptide). In some cases, the polypeptides of a composite provided herein can contain one or more aromatic amino acid residues (e.g., tyrosine, phenylalanine, or tryptophan) or one or more hydrophobic amino acids (e.g., alanine, isoleucine, leucine, or valine). Such aromatic amino acid residues can be arranged such that the polypeptide has the ability to interact (e.g., through hydrogen bonding or van der Waal forces) with a polysaccharide such cellulose. For example, a polypeptide of a composite provided herein can contain at least two tyrosine residues, preferably contiguous, such that the polypeptide can bind to cellulose. In some cases, a polypeptide can contain an amino acid sequence having aromatic amino acid residues as presented in Table 1.

TABLE 1
Possible amino acid sequences
having the ability to bind cellulose.

| | SEQ |
|---|---|
| Amino acid sequence | ID NO: |
| CSSVWGQCGGQNWSGPTCCASGSTCVYSNDYYSQCLP | 1 |
| YGQCGGIGYSGPTVCASGTTCQVLNPYYSQCL | 2 |
| TVPQWGQCGGIGYTGSTTCASPYTCHVLNPYYSQCY | 3 |
| TQSHYGQCGGIGYSGPTVCASGTTCQYLNPYYSQCL | 4 |
| CSSVWGQCGGQNWSGPTCCASGSTCVYSNDYYSQCL | 5 |
| TQTHWGQCGGIGYSGCKTCTSGTTCQYSNDYYSQCL | 6 |
| QQTVWGQCGGIGWSGPTNCAPGSACSTLNPYYAQCI | 7 |
| NDYYSQCL | 8 |
| NPYYSQCL | 9 |
| GGGGYYGGGG | 10 |
| GGGGYGYGGGG | 11 |
| GGGGYGGYGGGG | 12 |
| GGGGYGGGYGGGG | 13 |
| GGGGYYYGGGG | 14 |
| GGGGYYGGGYYGGGG | 15 |
| GGGGWYGGGG | 16 |
| GGGGYYSQCLGGGG | 17 |
| GGGGYYSQCLGGGG | 18 |
| QQQQYYQQQQ | 19 |
| QQQQYQYQQQQ | 20 |
| QQQQYQQYQQQQ | 21 |
| QQQQYQQQYQQQQ | 22 |
| QQQQYYYQQQQ | 23 |
| QQQQYYQQQYYQQQQ | 24 |
| QQQQWYQQQQ | 25 |
| RCEKDERFFSDKIAKYIPIQYVLSRYPSYGLNYYQQKPVALIN | 26 |
| NQFLPYPYYAKPAAVRSSAQILQWQVLSNTVPAKSCQAQPTT | |
| MARHPHPHLSF | |
| NQQLAYFYPQLF | 27 |
| PSGAWYYVPLGT | 28 |

In some cases, the polypeptides of a composite provided herein can contain one or more phosphorylated amino acid residues (e.g., phosphoserine, phosphothreonine, phosphotyrosine, or phosphohistidine). Such phosphorylated amino acid residues can be arranged such that the polypeptide has the ability to interact (e.g., ionic interactions) with a calcium-containing molecule (e.g., calcium phosphate or calcium carbonate) or a clay (e.g., kaolinite type, colloidal clays (e.g., bentonites), non-colloidal clays (e.g., china clay), other plastic clays (e.g., illite and montmorilionite), or metals or metal oxides or metal alloys exhibiting a positive charge or positive surface charge (e.g., Fe, Cu, Ni, TiO, Ti₂O₃, NiTi). For example, a polypeptide of a composite provided herein can contain at least two (e.g., at least three, at least four, at least five, at least six, at least seven, at least eight, at least nine, at least ten, at least 11, at least 12, at least 13, at least 14, or at least 15) phosphoserine residues within a span of about 30 contiguous amino acid residues such that the polypeptide can bind to a calcium-containing molecule. In some cases, a polypeptide can contain an amino acid sequence having phosphorylated amino acid residues as presented in Table 2.

TABLE 2
Possible amino acid sequences having the
ability to bind a calcium-containing molecule.

| | SEQ |
|---|---|
| | ID |
| Amino acid sequence | NO: |
| SS | N/A |
| SSS | N/A |
| SSSS | 29 |
| YY | N/A |
| YYY | N/A |
| YYYY | 30 |
| FF | N/A |
| FFF | N/A |
| FFFF | 31 |
| WW | N/A |
| WWW | N/A |
| WWWW | 32 |
| II | N/A |
| III | N/A |
| IIII | 33 |
| MEAESISSSEEIV | 34 |
| ELSKAIGSESTEDQA | 35 |
| MSLSIDVTSLPSISSSVYKNESFSTTSTISGKSIGRSEQYISPDA | 36 |
| EAFNKYMLSKSPEDIGPSDSASNDPLTSFSIRSNAVKTNADAGV | |
| SMDSSAQSRPSS DIGFDQMDF | |
| SLNKGIKIDATMDSSISISTTSKKEKSKQENKNKYKKCYPKIEA | 37 |
| ESDSDEYVLDDSDSDDGKCKNCKYKK | |
| ITSNLVPGFIGVSSSETFLSSSSTLST | 38 |
| TSSRSISSSTLYENHLVNDCTNF | |
| SVGYLIGHSIVPSSSSSSSSSSSSIVVPSSRCMLLQTEKNTSIIS | 39 |
| SLCSSSTDNLNYLNSSSPHLSNHNNLHHHHYRQQQ | |
| LELSDDDDESKASINETQPPQ | 40 |
| SYFWIP | 41 |
| SSYYFFWWIIPP | 42 |
| SSSYYYFFFWWWIIIPPP | 43 |
| SSSGGGYYYGGGFFFGGGWWWGGGIIIGGGPPP | 44 |
| SSSQQQYYYQQQFFFQQQWWWQQQIIIQQQPPP | 45 |
| GGGGSSSGGGG | 46 |
| QQQQSSSQQQQ | 47 |

In some cases, the polypeptides of a composite provided herein can contain one or more hydrophobic and/or aromatic amino acid residues (e.g., tyrosine, phenylalanine, tryptophan, isoleucine, or proline). Such hydrophobic and/or aromatic amino acid residues can be arranged such that the polypeptide has the ability to interact (e.g., hydrophobic interactions, van der Waals interactions, or hydrogen bonding) with a polyester (e.g., polyhydroxybutyrate or polylactic acid) or other polymers such as polyethylene or polystyrene. For example, a polypeptide of a composite provided herein can contain at least two (e.g., at least three, at least four, at least five, at least six, at least seven, at least eight, at least nine, at least ten, at least 11, at least 12, at least 13, at least 14, or at least 15) tyrosine or isoleucine residues within a span of about 45 (e.g., at least about 40, at least about 35, at least about 30, at least about 25, at least about 20, at least about 15, at least about 10, at least about 5, or at least about two) contiguous amino acid residues such that the polypeptide can bind to a polyester. In some cases, a polypeptide of a composite provided herein can contain at least two (e.g., at least three, at least four, at least five, at least six, at least seven, at least eight, at least nine, or at least ten) contiguous tyrosine residues, isoleucine residues, or a combination of tyrosine residues and isoleucine residues. In some cases, a polypeptide can contain an amino acid sequence having hydrophobic and/or aromatic amino acid residues as presented in Table 3.

TABLE 3
Possible amino acid sequences having the
ability to bind a polyester molecule.

| Amino acid sequence | SEQ ID NO: |
|---|---|
| AA | N/A |
| AAA | N/A |
| AAAA | 48 |
| YY | N/A |
| YYY | N/A |
| YYYY | 49 |
| MM | N/A |
| MMM | N/A |
| MMMM | 50 |
| VV | N/A |
| VVV | N/A |
| VVVV | 51 |
| LL | N/A |
| LLL | N/A |
| LLLL | 52 |
| WW | N/A |
| WWW | N/A |
| WWWW | 53 |
| II | N/A |
| III | N/A |
| IIII | 54 |
| FF | N/A |
| FFF | N/A |
| FFFF | 55 |
| GGGGLLLGGGG | 56 |
| QQQQLLLQQQQ | 57 |
| GGGGIIIGGGG | 58 |
| QQQQIIIQQQQ | 59 |
| GGGGAAAGGGG | 60 |
| QQQQAAAQQQQ | 61 |
| GGGGVVVGGGG | 62 |
| QQQQVVVGGGG | 63 |
| GGGGFFFGGGG | 64 |
| QQQQFFFQQQQ | 65 |
| GGGGWWWGGGG | 66 |
| QQQQWWWQQQQ | 67 |
| GGGGYYYGGGG | 68 |
| QQQQYYYQQQQ | 69 |
| GGGGIIIGGGGYYYGGGG | 70 |
| QQQQIIIQQQQYYYQQQQ | 71 |

In some cases, the polypeptides of a composite provided herein can contain one or more positively charged amino acid residues (e.g., lysine, arginine or histidine). Such positively charged amino acid residues can be arranged such that the polypeptide has the ability to interact (e.g., electrostatically or ionically) with cellulose, which exhibits a negative charge (e.g., a zeta potential of −10 to −20 mV over a pH range of about 5 to 11). For example, a polypeptide of a composite provided herein can contain at least two (e.g., at least three, at least four, at least five, at least six, at least seven, at least eight, at least nine, at least ten, at least 11, at least 12, at least 13, at least 14, or at least 15) lysine or arginine residues within a span of about 45 (e.g., at least about 40, at least about 35, at least about 30, at least about 25, at least about 20, at least about 15, at least about 10, at least about 5, or at least about two) contiguous amino acid residues such that the polypeptide can bind to cellulose. In some cases, a polypeptide of a composite provided herein can contain at least two (e.g., at least three, at least four, at least five, at least six, at least seven, at least eight, at least nine, or at least ten) contiguous lysine residues or arginine residues or a combination of lysine residues and arginine residues. In some cases, a polypeptide can contain an amino acid sequence having hydrophobic and/or aromatic amino acid residues as presented in Table 4.

TABLE 4
Possible amino acid sequences having the
ability to bind a cellulose molecule.

| Amino acid sequence | SEQ ID NO: |
|---|---|
| KK | N/A |
| KKK | N/A |
| KKKK | 72 |
| RR | N/A |
| RRR | N/A |
| RRRR | 73 |

The polypeptides of a composite provided herein can include any number of amino acid residues. For example, a polypeptide can have between four and 1500 amino acid residues (e.g., between 10 and 1500, between 15 and 1200, between 25 and 1000, between 30 and 750, between 50 and 500, between 100 and 500, between 150 and 500, between 175 and 500, between 180 and 500, between 50 and 400, between 50 and 300, between 50 and 200, or between 100 and 200). As described herein, a polypeptide of a composite provided herein can include a region having the ability to interact with a polysaccharide (e.g., cellulose). Such a region can have any number of amino acid residues. For example, the length of a region having the ability to interact with a polysaccharide can be between two and 300 amino acid residues (e.g., between two and 250, between two and 200, between two and 150, between two and 100, between two and 50, between two and 25, between two and 15, between three and 250, between four and 250, between five and 250, between six and 250, between seven and 250, between eight and 250, between nine and 250, between ten and 250, between 15 and 250, between 25 and 250, or between 50 and 250 amino acid residues).

In some cases, a polypeptide of a composite provided herein can include a region having the ability to interact with a calcium-containing molecule (e.g., HA). Such a region can have any number of amino acid residues. For example, the length of a region having the ability to interact with a calcium-containing molecule can be between two and 300 amino acid residues (e.g., between two and 250, between two and 200, between two and 150, between two and 100, between two and 50, between two and 25, between two and 15, between three and 250, between four and 250, between five and 250, between six and 250, between seven and 250, between eight and 250, between nine and 250, between ten and 250, between 15 and 250, between 25 and 250, or between 50 and 250 amino acid residues).

In some cases, a polypeptide of a composite provided herein can include a region having the ability to interact with a polyester (e.g., polylactic acid) or other polymers such as polyethylene or polystyrene. Such a region can have any number of amino acid residues. For example, the length of a region having the ability to interact with a polyester can be between two and 300 amino acid residues (e.g., between two and 250, between two and 200, between two and 150, between two and 100, between two and 50, between two and 25, between two and 15, between three and 250, between four and 250, between five and 250, between six and 250, between seven and 250, between eight and 250, between nine and 250, between ten and 250, between 15 and 250, between 25 and 250, or between 50 and 250 amino acid residues).

In some cases, the polypeptides of a composite provided herein can be designed to contain any number of regions and/or any combination of regions. For example, common molecular cloning techniques can be used to engineer nucleic acid to encode a polypeptide having five regions with the ability to interact with calcium-containing molecules and two regions with the ability to interact with polysaccharides. In some cases, such regions can be designed to be separated by linker sequences (e.g., a stretch of two to 20 glycine residues, a stretch of three to 20 glycine residues, a stretch of four to 20 glycine residues, a stretch of five to 20 glycine residues, or a stretch of six to 20 glycine residues, a stretch of two to 20 glutamine residues, a stretch of three to 20 glutamine residues, a stretch of four to 20 glutamine residues, a stretch of five to 20 glutamine residues, or a stretch of six to 20 glutamine residues).

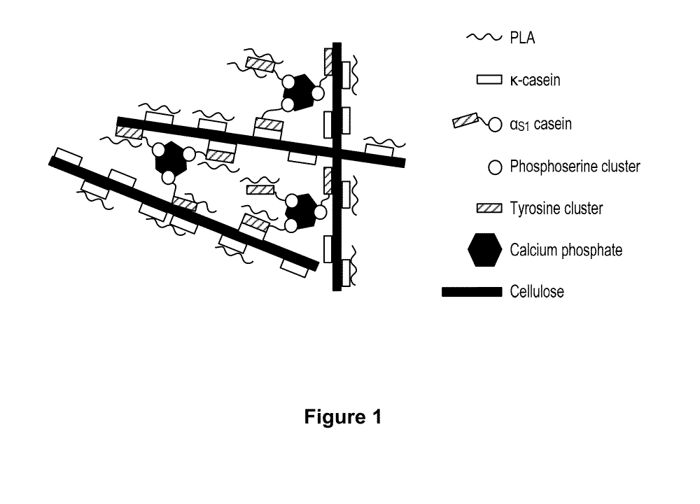

The polypeptides of a composite provided herein can have any amino acid sequence. For example, a polypeptide of a composite provided herein can have (a) one or more amino acid sequences such that the polypeptide has the ability to interact with one or more polysaccharides (e.g., cellulose), (b) one or more amino acid sequences such that the polypeptide has the ability to interact with one or more calcium-containing molecules (e.g., calcium phosphate or calcium carbonate) or clays (e.g., kaolinite type, colloidal clays (e.g., bentonites), non-colloidal clays (e.g., china clay), or other plastic clays (e.g., illite or montmorilionite), (c) one or more amino acid sequences such that the polypeptide has the ability to interact with one or more polyester molecules (e.g., polylactic acid or polyhydroxybutyrate) or other polymers such as polyethylene or polystyrene, or (d) any combination thereof. Such amino acid sequences can be found in naturally-occurring polypeptides or can be designed. In one embodiment, the polypeptides of a composite provided herein can have the ability to bind polysaccharides and non-polypeptide, non-polysaccharide molecules. For example, a composite provided herein can contain casein polypeptides having the ability to bind cellulose, polylactic acid, calcium phosphate, and other casein polypeptides (FIG. 1).

In some cases, a polypeptide of a composite provided herein can include one or more sequences set forth in Table 1, one or more sequences set forth in Table 2, one or more sequences set forth in Table 3, or any combination thereof. Examples of polypeptides that have the ability to interact with polysaccharides, calcium-containing molecules, and polyesters include, without limitation, casein polypeptides from any species (e.g., bovine, monkey, human, or goat), RABAB polypeptides (e.g., human RABAB polypeptides such as ID No. 2a5j from the RCSB protein data bank), neurexin 1beta polypeptides (e.g., rat neurexin 1beta such as ID No. 2r1b or ID No. 2r1d from the RCSB protein data bank), and SusD polypeptides (e.g., Bacteriodes thetaiotaomicron VPI-5482 SusD polypeptides such as ID No. 3iv0 from the RCSB protein data bank (see also, GenBank Accession No. NP_{—}809186.1 or GI No. 29345683), glucoamylase polypeptides (e.g., Saccharomycopsis fibuligera glucoamylase polypeptides such as ID No. 2f6d from the RCSB protein data bank), or fragments thereof.

A casein polypeptide can be a multi-subunit casein protein or can be a single casein subunit polypeptide such as a αₛ₁-, αₛ₂-, β-, or κ-casein subunit. The amino acid sequence of a casein polypeptide can be as set forth in GenBank® as follows in Table 4.

TABLE 4
Casein subunits polypeptides.

| Casein subunit | GI # | Accession # |
|---|---|---|
| Bos taurus (cow) | | |
| alphaₛ1 | 217533 | BAA00313 |
| alphaₛ2 | 54144010 | CAH61065 |
| beta | 148767917 | ABR10906 |
| kappa | 2801548 | AAB97519 |
| Homo sapien | | |
| alphaₛ1 | 854086 | CAA55185 |
| alphaₛ2 | — | — |
| beta | 288098 | CAA39270 |
| kappa | 29676 | CAA47048 |
| Ovis aries | | |
| alphaₛ1 | 57526469 | NP_001009795 |
| alphaₛ2 | 732894 | CAA26983 |
| beta | 1211 | CAA34502 |
| kappa | 1840105 | AAB47262 |
| Capra hircus (goat) | | |
| alphaₛ1 | 22796155 | CAD45345 |
| alphaₛ2 | 448348 | 1916449A |
| beta | 4499833 | CAB39313 |
| kappa | 978 | CAA43174 |

In some cases, the polypeptides of a composite provided herein can contain one or more identifiable motif structures (e.g., an SSS motif structure or a YYSQCL motif structure).

In some cases, a polypeptide of a composite provided herein can be obtained from naturally-occurring starting material or can be obtained recombinantly. For example, when casein polypeptides are used, milk can be used as a starting material to obtain large quantities of the casein polypeptides. In such cases, the milk can be obtained from any appropriate species including, without limitation, cows, pigs, goats, sheep, monkeys, and humans. Any appropriate cloning techniques and heterologous expression system technologies can be used to obtain a polypeptide recombinantly. For example, nucleic acid encoding a naturally-occurring κ-casein polypeptide or a polypeptide designed to contain one or more of the regions described herein can be inserted into an expression vector (e.g., a mammalian or bacterial expression vector) for expression in desired cells (e.g., mammalian or bacterial cells). Examples of such vectors include, without limitation, viral vectors and non-viral vectors. Once inserted into a cell, the vector can drive expression of large quantities of the encoded polypeptide, which can be purified using common purification techniques. For example, affinity chromatography can be used to purify recombinantly produced polypeptides. In some cases, an enzyme such as a protease can be used to cleave a particular polypeptide, and one or more of the cleavage products can be obtained and used to make a composite provided herein. For example, κ-casein, after binding to cellulose, can be cleaved by the enzyme chymosin to leave a bound polypeptide containing hydrophobic and aromatic amino acids that can be used to bind to polysaccharides and/or organic polymers.

A composite provided herein can include one or more polysaccharides (e.g., cellulose). Such polysaccharides can be homopolysaccharides or heteropolysaccharides and can contain two or more monosaccharide residues (e.g., glucose residues, mannose residues, galactose residues, fructose residues, arabinose residues and xylose residues). For example, the polysaccharides of a composite provided herein can include between 50 and 20,000 monosaccharide residues (e.g., between 50 and 15,000, between 50 and 10,000, between 50 and 5,000, between 50 and 2500, between 50 and 2,000, between 50 and 1,500, between 50 and 1,000, between 50 and 500, between 100 and 20,000, between 500 and 20,000, between 1,000 and 20,000, between 2,000 and 20,000, between 500 and 15,000, between 1,000 and 15,000, between 2,000 and 15,000, between 500 and 2,000, or between 500 and 1,000 monosaccharide residues). The polysaccharides of a composite provided herein can include any type of glycosidic bond (e.g., β-1,4 linkages, α-1,4 linkages, β-1,6 linkages, and α-1,6 linkages) or any combination of glycosidic bonds. Examples of polysaccharides that can be used to make a composite provided herein include, without limitation, cellulose (e.g., plant, insect, or microbial cellulose), starch, chitin, fructan, amylose, amylopectin, glycogen, xanthan, mannan, galactomamman, xylan, glucuronoxylan, arabinoxylan, glucomannan, xyloglucan, glycosaminoglycans, modified starches, modified amylopectin, modified amylose, chitosan, guar gum, modified guar gum, locust bean gum, tara gum, konjac gum, konjac flour, fenugreek gum, mesquite gum, aloe mannans, modified cellulose such as carboxyalkylated cellulose and carboxymethyl cellulose, oxidized polysaccharides, sulfated polysaccharides, cationic polysaccharides, pectin, arabic gum, karaya gum, xanthan, kappa, iota or lambda carrageenans, agar-agar, alginates, guar gum, tara gum, locust bean gum, konjac, mesquite gum, and fenugreek extracts.

Polysaccharides can be obtained in the form of a liquid, gel, powder, matrix, or sphere-like particle. For example, cellulose can be used as described herein in the form of microcrystalline cellulose, microfibrillated cellulose, or hydrolyzed cellulose nanofibers or nanowhiskers, or sphere-like cellulose produced by bacteria including Acetobacter xylinum. Cellulose in sphere-like form can range in size from about 50 μm to about 25000 μm (e.g., 200 μm to 1000 μm, 500 μm to 5000 μm, or 1000 μm to 10000 μm).

Polysaccharides can be obtained from a naturally-occurring starting material or can be produced synthetically. For example, the polysaccharides of a composite provide herein can be obtained from plants (e.g., grasses and trees), animals (e.g., tunicates), or microbes (e.g., Acetobacter xylinum bacteria). In some cases, the polysaccharides of a composite provide herein can be obtained commercially. For example, various grades of cellulose can be obtained from paper and pulp manufacturers such as International Paper, Georgia Pacific, or Weyerhaeuser, or distributors such as Fluka, Sigma Aldrich, and other companies.

In some cases, a polysaccharide provided herein can exhibit various degrees of alignment. For example, cellulose fibrils can be aligned using a magnetic field, an electric field (e.g., a DC or AC electric field), an electromagnetic or optical field, or using fluid flow, where the long axis (along the α-1,4 glucan chain) of the fibrils are generally parallel. Such a configuration can be achieved by applying an electric field to a solution of cellulose fibers or to an active growing culture of microbes (such as the bacteria Acetobacter xylinum) producing cellulose. Such an arrangement can improve the physical properties of the cellulose or any cellulose containing materials and can be used in tissue regeneration applications where growing cells need to grow primarily in one dimension (e.g., along the fiber length). An example of such tissue is nerve tissue (e.g., spinal cord tissue after a break where the break is larger than about 10 μm to about 100 μm).

In some cases, the cellulose provided herein can include nanofibers of cellulose (e.g., nanofibers measuring 2 nm to about 35 nm in diameter, 50 nm to 50,000 nm in length). The nanofibers can be created using microbes in a culture. The culture conditions can be such the temperature is cycled. For example, the temperature can periodically exceed the thermal stability of the cellulose synthase complex. In some cases, the cellulose nanofibers can be produced by culturing the bacteria Acetobacter xylinum in an agitated culture where the culture temperature is cycled as follows: the culture is maintained at 30° C. for 10 minutes to 12 hours, then the temperature is increased to 36° C. to 44° C. (e.g., between 40° C. to 42° C.) and held there for 1 minute to 6 hours (e.g., 5 minutes to 60 minutes). The high temperature can disrupt cellulose synthesis. The 30° C. temperature culture can allow the cellulose to grow normally.

A composite provided herein can include one or more non-polypeptide, non-polysaccharide molecules such as calcium-containing molecules, polyesters, or petroleum derived polymers. Examples of calcium-containing molecules include, without limitation, calcium phosphate, HA, calcium carbonate, calcium hydroxide, calcium hypophosphite, calcium oxalate, calcium sulfate, calcium lactate, calcium fluoride, calcium silicate, calcium periodate, calcium sulphate, calcium aspartate, calcium carbide, calcium chloride, calcium cyclamate, calcium gluconate, calcium hypochlorite, calcium permanganate, calcium phosphide, calcium stearate, and calcium sulfate. Calcium-containing molecules can be obtained in any appropriate form such as a liquid, solid, powder, or granule. For example, calcium phosphate can be obtained in the form of an amorphous powder. Calcium-containing molecules can be obtained from naturally-occurring starting materials, can be synthetically produced, or can be obtained commercially. For example, HA can be obtained commercially from Sigma Aldrich. In some cases, a non-polypeptide, non-polysaccharide molecules can be a clay such as kaolin, bentonite, and other colloidal, non-colloidal, and plastic clays.

Examples of polyester and other polymer molecules include, without limitation, polylactic acid, poly(lactic-co-glycolic acid), polyhydroxybutyrate, polycaprolactone, polybutylene succinate, polyethylene, and polystyrene. Such polymers can be obtained in any appropriate form such as a liquid, solid, powder, or granule. For example, polyesters can be obtained in the form of granules or pellets. Polyester molecules can be obtained from a naturally-occurring starting material, can be synthetically produced, or can be obtained commercially. For example, polylactic acid can be synthetically produced from lactic acid using any appropriate method including, without limitation, oligomerization, dimerization, and/or ring-opening polymerization.

In some cases, a composite provided herein can contain polymers such as lignin either purified from plant feed stocks or formed during the growth of the plant. For example, polypeptides described herein can be used to bind lignin to cellulose, other polysaccharides, or non-polysaccharide, non-polypeptide molecules. Expression of such polypeptides in the plant cell wall can provide composite materials with improved properties. For example, the expression of polypeptides in the plant cell wall which bind lignin to cellulose could improve the mechanical properties of the plant material, i.e., a wood material with improved tensile strength, compression strength, and/or bending strength. Such a polypeptide can be useful for disassembling the plant cell wall by providing a linker between lignin and other plant cell wall polysaccharides that can be easily cleaved with a protease such as protease K. Improved processes for the disassembly of the plant cell such as treatment with proteases on genetically modified plant material where the polypeptides are expressed and incorporated into their cell walls can provide improved purification processes where cellulose or other plant polysaccharides can be more easily separated from lignin. Such methods and materials can be used for subsequent chemical or biofuel production from plant materials.

A composite provided herein containing polypeptides attached to polysaccharides and non-polypeptide, non-polysaccharide molecules can include any of the following combinations of items: (a) cellulose, casein, and calcium phosphate; (b) cellulose, casein, and polylactic acid; (c) cellulose, casein, calcium phosphate, and polylactic acid; (d) cellulose, casein, and calcium carbonate; (e) cellulose, casein, and polyhydroxybutyrate; (f) cellulose, α_{S1}-casein, calcium phosphate, and polylactic acid; (g) cellulose, α_{S2}-casein, calcium phosphate, and polylactic acid; (h) cellulose, κ-casein, calcium phosphate, and polylactic acid; (i) cellulose, β-casein, calcium phosphate, and polylactic acid; (j) cellulose, α_{S1}-casein, calcium carbonate, and polylactic acid; (k) cellulose, α_{S2}-casein, calcium carbonate, and polylactic acid; (1) cellulose, κ-casein, calcium carbonate, and polylactic acid; (m) cellulose, β-casein, calcium carbonate, and polylactic acid; (n) cellulose, hydrophobic portion of κ-casein (amino acids 1-105), calcium phosphate, and polylactic acid; (o) cellulose, hydrophobic portion of κ-casein (amino acids 1-105), calcium carbonate, and polylactic acid; (p) cellulose, hydrophobic portion of κ-casein (amino acids 1-105), calcium phosphate, and polylactic acid; (q) cellulose, casein, and polyethylene; (r) cellulose, casein, calcium phosphate, and polyethylene; (s) cellulose, α_{S1}-casein, and polyethylene; (t) cellulose, α_{S2}-casein, and polyethylene; (u) cellulose, β-casein, and polyethylene; (v) cellulose, κ-casein, and polyethylene; (w) cellulose, α_{S1}-casein, and polystyrene; (x) cellulose, α_{S2}-casein, and polystyrene; (y) cellulose, β-casein, and polystyrene; (z) cellulose, κ-casein, and polystyrene; (aa) cellulose, casein, calcium phosphate, and polystyrene; (ab) cellulose, α_{S1}-casein and κ-casein, calcium carbonate, and polyhydroxybutyrate; (ac) cellulose, α_{S1}-casein and κ-casein, calcium phosphate, and polyhydroxybutyrate; (ad) cellulose, α_{S1}-casein and κ-casein, calcium phosphate, and polylactic acid; and (ae) cellulose, α_{S1}-casein and hydrophobic portion κ-casein (amino acids 1-105), calcium phosphate, and polylactic acid. In some cases, a composite provided herein can be designed to include any of the following combinations of items (a) casein and polylactic acid; (b) casein, calcium phosphate, and polylactic acid; (c) α_{S1}-casein and polylactic acid; (d) α_{S2}-casein and polylactic acid; (e) β-casein and polylactic acid; (f) κ-casein and polylactic acid; (g) hydrophobic portion κ-casein (amino acids 1-105), calcium phosphate, and polylactic acid; (h) casein and polyethylene; (i) casein, calcium phosphate, and polyethylene; (j) α_{S1}-casein and polyethylene; (k) α_{S2}-casein and polyethylene; (1) β-casein and polyethylene; (m) κ-casein and polyethylene; or (n) hydrophobic portion κ-casein (amino acids 1-105), calcium phosphate, and polyethylene.

In some cases, a composite provided herein can have between about 1 mg and about 1 kg of a polypeptide, between about 1 mg and about 1 kg of a polysaccharide, and between about 1 mg and about 1 kg of a non-polypeptide, non-polysaccharide molecule. In some cases, a composite provided herein can have between 0.5 and 99 wt % of a polypeptide, between 0.5 and 99 wt % of a polysaccharide, and between 0.5 and 99 wt % of a non-polypeptide, non-polysaccharide molecule. For example, a composite provided herein can contain polypeptides, polysaccharides, and non-polypeptide, non-polysaccharide molecules in the weight percentages set forth in Table 5.

TABLE 5
Weight percentages of solid ingredients within a composite.

| | | | non-polypeptide, |
|---|---|---|---|
| | Polypeptide | Polysaccharide | non-polysaccharide |
| Composite # | (wt %) | (wt %) | molecule (wt %) |
| 1 | 1% | 98% | 1% |
| 2 | 1% | 94% | 5% |
| 3 | 1% | 89% | 10% |
| 4 | 2% | 97% | 1% |
| 5 | 2% | 93% | 5% |
| 6 | 2% | 88% | 10% |
| 7 | 5% | 94% | 1% |
| 8 | 5% | 90% | 5% |
| 9 | 5% | 85% | 10% |
| 10 | 5% | 80% | 15% |
| 11 | 5% | 75% | 20% |
| 12 | 10% | 89% | 1% |
| 13 | 10% | 85% | 5% |
| 14 | 10% | 80% | 10% |
| 15 | 10% | 75% | 15% |
| 16 | 10% | 70% | 20% |
| 17 | 10% | 40% | 50% |
| 18 | 10% | 15% | 75% |

In one embodiment, a composite containing a polypeptide attached to cellulose and a calcium-containing molecule can be synthesized by obtaining microbial cellulose in a static or agitated culture using bacteria such as Acetobacter xylinum. The culture can be washed with a basic solution (e.g., sodium hydroxide) to remove bacteria cells and media compounds. A polypeptide (e.g., whole milk bovine casein, α_{S1}-casein, α_{S2}-casein, β-casein, κ-casein, or the hydrophobic portion of κ-casein, amino acids 1-105)) can be contacted with the microbial cellulose. The resulting mixture can be mixed for greater than or equal to 10 minutes (e.g., 30 minutes) to allow binding. The mixture can be washed repeatedly (e.g., 3 to 4 times) for 15 minutes on a rotor to remove unbound casein. A calcium phosphate solution (e.g., hydroxyapatite) can be introduced to the mixture. The solution can contain calcium phosphate particles in the size range of 5 nm to 1 micron. Any excess unbound calcium phosphate particles can be washed out.

In another embodiment, a composite containing a polypeptide attached to cellulose and a calcium-containing molecule can be synthesized by obtaining microbial cellulose in a static or agitated culture using bacteria such as Acetobacter xylinum. The culture can be washed with a basic solution (e.g., sodium hydroxide) to remove bacteria cells and media compounds. A polypeptide (e.g., whole milk bovine casein or α_{S1}-casein) can be introduced into a calcium phosphate solution (e.g., hydroxyapatite) once a pH (e.g., a pH between 7.0 and 11.0 or a basic pH between 9.5 to 11.0) is achieved using NaOH and water. The solution can contain calcium phosphate particles in the size range of 5 nm to 1 micron. The solution can be mixed for 30 minutes to allow binding. The particles can be centrifuged repeatedly (e.g., 3-4 times) and washed to remove excess unbound casein. The functionalized calcium phosphate particles can be mixed with the microbial cellulose and rotored for 10-30 minutes to allow binding. The mixture can be washed repeatedly (e.g., 3-4 times) for 15 minutes on a rotor to remove excess unbound functionalized calcium phosphate particles.

The composites provided herein can be designed to be in the form of a matrix, microsphere, coated fabric, liquid, hydrogel, dry coating, powder, foam, paste, cream, or injectable. In some cases, a composite provided herein can be in a form appropriate for injection into a human. For example, a composite provided herein can be a sterile hydrogel formulation. Such sterile hydrogel formulations can be used in combination with a syringe. In such cases, a unit dose can be provided in the syringe such that between about 0.5 mL to about 500 mL of a composite provided herein is delivered (e.g., between about 1 mL to about 50 mL, between about 2 mL to about 25 mL, between about 2 mL to about 10 mL, or between about 1 mL to about 10 mL). In some cases, a composite provided herein can be formulated into a bioabsorbable material. For example, a composite provided herein containing HA can be formulated into a patch that can be used as an osteoinductive tissue scaffold. Such patches can be used to promote bone growth within mammals (e.g., humans).

In some cases, a composite provided herein can be formulated into a tissue scaffold specifically engineered to guide the direction of tissue growth. For example, a composite provided herein can contain aligned cellulose fibers.

In some cases, a composite provided herein can be formulated to be a cosmetic compound. For example, a composite provided herein containing cellulose and casein, or cellulose, casein, and a mineral or clay can be used as a facial cream or paste. In this case, the cellulose can help provide a final material coating where the coating smoothens the surface of the skin, reducing the appearance of wrinkles. The casein polypeptide containing hydrophobic amino acids can make the cellulose, minerals, or clay compatible or have an affinity for, the lipids or oily surface of the skin or other biological components positioned on the skin surface.

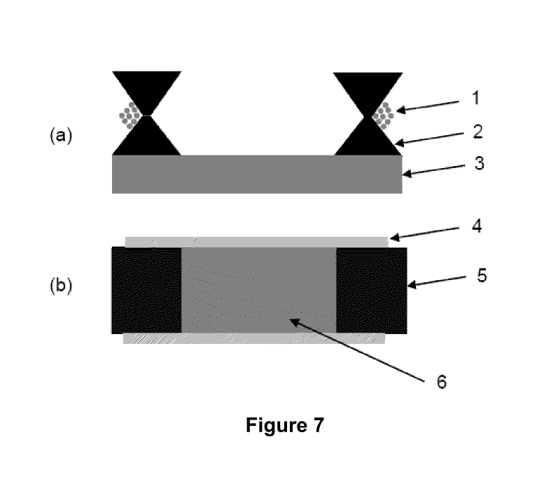

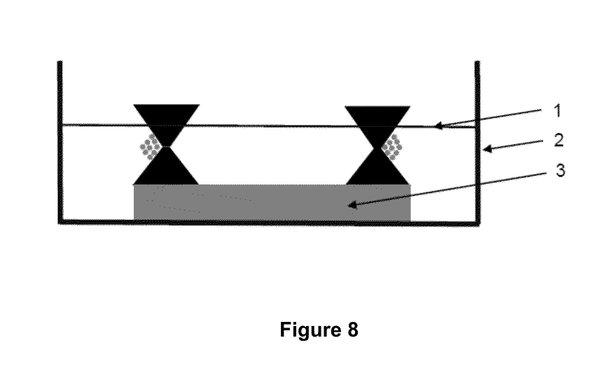

In some cases, a polysaccharide or composite provided herein can be used as a tissue regeneration material for applications such as skin, bone, cartilage, nerve, organ, animal muscle, or other tissues. The polysaccharide or composition can be formed with a three dimensional structure. For example, the polysaccharide or composition can contain microbially produced polysaccharides where the shape of the polysaccharide material is formed using a template material, i.e., the microbially produced polysaccharide grows over and/or in between features of the template material to form a three dimensional structure (see, e.g., FIGS. 7 and 8). The template material can be degradable such that either as the polysaccharide is microbially produced or after the polysaccharide is formed, the template material can be removed. Non-microbially produced polysaccharides or polysaccharide compositions can be formed with a three dimensional structure using a template or degradable template. One example of a degradable template is poly(lactic-co-glycolic acid). This material can degrade in water. The degradation rate can be modified by changing the pH of a solution in which the material can be placed. For example, the degradation rate of poly(lactic-co-glycolic acid) can be increased over that of pure water by increasing the pH to a value of 8-12. The poly(lactic-co-glycolic acid) can be in the shape of spheres, fibers, or particles with a distribution of dimensions, i.e., diameters of 5 nanometers to 500 microns or in the case of fibers a diameter of 5 nanometers to 500 microns and a length of 100 nanometers to 10 millimeters or more. In some cases, a composite provided herein can be used to produce an environmental remediation material, such as a material used to absorb oil spills. Hydrophobicity created by the incorporation of polypeptides that contain hydrophobic residues can absorb more oil, especially when combined with microbial cellulose, which can exhibit extensive porosity and high surface area. One particular example is the use of the hydrophobic portion of κ-casein (called para-kappa casein, residues 1-105). In some cases the material can be used to absorb metal and metal alloy ions through binding to the phosphorylated residues of the polypeptide.

In some cases, a composite provided herein can be used as a food product. For example, cellulose containing hydrophobic polypeptides can interact or bind with lipids, fats, or fatty acids. Since cellulose is not digestible by humans naturally, fats associated with the cellulose-polypeptide material can pass through the body without being digested resulting in reduced fat consumption. This process can be improved using nanodimensional cellulose such as cellulose nanofibers or cellulose nanowhiskers that exhibit increased surface area and mobility during digestion processes. In addition, cellulose in combination with casein can provide desirable rheological, texture, or taste modification to processes or engineered foods. In one example, the cellulose or composites provided herein can be used as a tissue scaffold for the growth of animal cells. For example, cellulose tissue scaffolds can be used for the growth of animal muscle cells that can be used for producing meat products. Cellulose is particularly beneficial in these cases since it can be consumed with the meat product as it is edible.

In some cases, a composite provided herein can be used as a drug delivery device for enhancement of calcium delivery or bio-activity. For example, cellulose porosity and bio-compatibility after functionalized with casein can be injected with a pharmaceutical for controlled release in the body. Since cellulose is not digested by humans, casein hydrolysis in the digestive tract will allow for a timed-release of incorporated drug(s). In addition, an edible cellulose-casein-calcium supplement can provide desirable delivery or bio-availability of calcium as salivary enzymes break down the casein releasing the calcium for dental bone deposition or nutrient delivery.

In some cases, a composite provided herein can be used as a component in chewing gum or a chewable candy that can be completely edible (e.g., it can be chewable such that it degrades during chewing and is ultimately consumed and not discarded). Microbial cellulose, including statically grown microbial cellulose, can have mechanical properties that allow for its use as a chewable material, or partially chewable material, containing other food compounds providing flavor, nutritional ingredients such as vitamins, or drug/biomedical compounds useful for therapeutic applications, disease treatment, or health improvement.

In some cases, a composite provided herein can be used as an improved fire retarding insulation, wallboard, filler, or clothing material. The incorporation of calcium containing molecules or clays, that exhibit good thermal degradation properties (e.g., high thermal degradation properties), onto the surface of cellulose materials, can improve the fire resistance and thermal degradation properties of the material.

In some cases, a composite provided herein can be used as a foam where the stability of the foam can be improved through interactions of the polypeptides including disulfide bonding or ionic interactions including those associated with the phosphorylated amino acids which can bind through intermediate positively charged ions, molecules, or particles such as calcium, calcium phosphate, or metal ions. The production of the foam can be accomplished by introducing the ions, molecules, or particles during the foaming process. The polypeptides can include αS1-casein, αS2-casein, and β-casein polypeptides. Other polypeptides include those described herein as well as those that contain more than three hydrophobic amino acids and more than three phosphorylated amino acids per 10-20 amino acids.

In some cases, a composite provided herein can be used as an acoustic device such as an audio speaker diaphragm or micro- or nano-scale electromechanical system device. In the case where aligned cellulose nanofibers are implemented (e.g., cellulose nanofibers measuring 2 nm to about 35 nm diameter, 50 nm to 50,000 nm length where the long axis of the fibers are generally parallel), the material can also exhibit an improved piezoelectric response (e.g., a resulting mechanical stress in the cellulose nanofibers resulting from an electric field or electric potential across the cellulose nanofiber or vice-versa). In this case, the aligned cellulose content can be greater than 60% where the free condensed unbound water content is less than 30%.

In some cases, a composite provided herein can be used to produce wood products (e.g., paper, photographic paper, cardboard, particle board, fiber board, wood chip board, packaging material, or a laminated, coated, or joined material). Wood products containing a composite provided herein can have an extended life and increased recyclability thus reducing the consumption and energy requirements associated with processing such wood products. For example, the composites provided herein can be used to improve the mechanical properties of paper and/or reduce the fiber content in processed wood products. In some cases, a composite provided herein can be in a form appropriate for coatings other materials. For example, a composite provided herein can be a clay formulation. Such clay formulation can be used to coat wood processed products such as paper, packaging, cardboard, particle board, or solid wood products.

In some cases, a composite provided herein can be used to reinforce concrete or cement. For example, a sheet that includes cellulose fiber, or a sheet that includes polymer fibers such as polylactic acid, polyethylene, and polystyrene, in conjunction with the polypeptides disclosed herein, including those including hydrophobic, aromatic, or positively charged amino acid residues, can be incorporated into cement or concrete as a structural reinforcement with improved mechanical properties such as improved tensile strength or strain at break. This includes the implementation of these sheets in a layered fashion, e.g., the sheets can be layered within the cement or concrete material. Such layers can be numerous, e.g., 1-10 per millimeter of thickness. Such concrete materials can be in the form of sheets measuring 5-50 mm thick and of any width and length, e.g., 6 inches to 20 feet. Such sheets can be used in several applications including structural and cosmetic siding, roofing, and flooring. In some cases, the cellulose, polylactic acid, polyethylene, or polystyrene fibers can be incorporated into the bulk not as a sheet but as an additive, where such fibers can be modified with the polypeptides described herein, including those having hydrophobic, aromatic, and/or positively charged amino acid residues.

In some cases, a composite provided herein can be in the form of a coating, or a composite provided herein can be a coated material. The coating can contain two or more oppositely charged polymers, polypeptides, or polysaccharides, in any combination. In some cases, a coating can improve the dry and wet mechanical strength of the substrate material, which can be a composite as described herein. In some cases, a coating can be made principally of cellulose and can have improved liquid barrier properties. An improved composite provided herein can be used for many applications including, without limitation, food packaging, shipping containers, backing for insulating material, or construction materials.

The invention will be further described in the following examples, which do not limit the scope of the invention described in the claims. In addition, the following examples do not limit further combinations of the materials described herein and other compositions capable of being created based on the inventions herein.

### EXAMPLES

### Example 1

### Producing Composite Containing Casein Polypeptides, Cellulose, and Calcium-Containing Molecules Such as HA or Calcium Carbonate

### Method #1:

Whole milk bovine casein (or purified casein subunit) is added to cellulose in an aqueous solution at a pH of 7-11 (adjusted through the addition of sodium hydroxide) and mixed on a rotor for at least 10 minutes (e.g., 10-30 minutes) at 10 RPM to 50 RPM. The ratio of the casein to cellulose is 1:200, 1:100, 1:50, 1:25, 1:10, 1:5, or 1:2. The solid content is 0.5%, 1%, 2%, 3%, 4%, 5%, 10%, or 25%. This cellulose is cellulose derived from plants (including trees) or from bacteria such as Acetobacter xylinum. This cellulose is in the form of a sheet or pellicle obtained from static bacteria cultures (about 0.5 mm to 25 mm thick), a mixture or mass of cellulose obtained from agitated bacteria cultures, in nanowhisker or nanofiber form (fibers measuring 2 nm to 35 nm diameter, 50 nm to 50000 nm length) produced via acid hydrolysis or other known processes, or in pellet or sphere form (roughly 100 μm to 20 mm in diameter) obtained from, for example, Acetobacter xylinum strain ATCC 700718 cultured in a 250 mL flask in with 100 mL of media in an orbital shaking incubator at 175 RPM and at 30° C. Cellulose is aligned cellulose where the cellulose fibers are aligned in an AC electric field. In some cases, the aligned cellulose is aligned in an electric field during its production in a culture of Acetobacter xylinum. Cellulose can be freeze-dried and dehydrated. In such cases, it can be rehydrated in the aqueous solution. This aqueous solution can contain the casein, promoting more complete or efficient incorporation of the casein onto the cellulose throughout the sample.

The cellulose is washed by centrifuge or straining 2-3 times to remove excess casein. HA (particle diameters 2 nm to 2 μm) is added and mixed on a rotor for at least 10 minutes (e.g., 10-30 minutes) at 10 RPM to 50 RPM. The weight ratio of the casein and the cellulose mixture to HA is 20:1, 10:1, 5:1, 2:1, 1:1, 1:2, 1:5, 1:10, or 1:20. The cellulose is washed by centrifuge or straining 2-3 times to remove excess HA. It is then freeze dried and sealed in a package with a desiccant or in dry air or nitrogen to preserve samples.

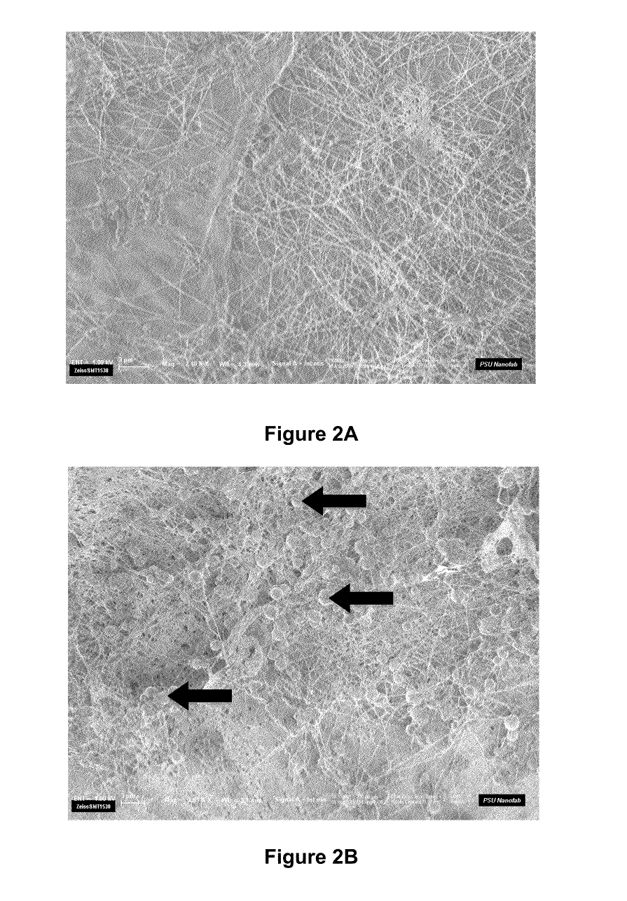

FIG. 2B is a scanning electron microscope image of a cellulose-casein-HA composite made as described in method #1. The spherical features are HA.

In some cases, aqueous solutions containing the HA-casein-cellulose material can be further modified by the incorporation of another material to form a gel-like composition. This is accomplished by adding the cellulose, casein, and HA mixture to a mixture of starch, chitosan, or carboxylmethylcellulose in an aqueous solution where the ratio of the content of the starch, chitosan, or carboxylmethylcellulose to total water content is 1:200, 1:100, 2:100, 3:100, 4:100, 5:100, or 10:100.

### Method #2:

Whole milk bovine casein (or purified casein subunit) is added to HA (particle diameters 2 nm to 2 μm) in a solution at a pH of 7-11 (adjusted through the addition of sodium hydroxide) and mixed on a rotor for at least 10 minutes (e.g., 10-30 minutes) at 10 RPM to 50 RPM. The weight ratio of the casein to HA is 1:100, 1:50, 1:25, 1:10, 1:2, 1:1, 2:1, 10:1, 25:1, 50:1, or 100:1. The solid content is 0.5%, 1%, 2%, 3%, 4%, 5%, 10%, or 25%. If desired, the HA is wash to remove excess casein 2-3 times by centrifugation. Cellulose is added to the solution. The final weight ratio of the casein and HA mixture to cellulose is 1:25, 1:10, 1:5, 1:2, 1:1, 2:1, 5:1, 10:1, or 25:1. This cellulose is cellulose derived from plants (including trees) or from bacteria such as Acetobacter xylinum. The cellulose can be in the form of a sheet or pellicle obtained from static bacteria cultures (about 0.5 mm to 25 mm thick), a mixture or mass of cellulose obtained from agitated bacteria cultures, in nanowhisker or nanofiber form (fibers measuring 2 nm to 35 nm diameter, 50 nm to 50000 nm length) produced via acid hydrolysis processes, or in pellet or sphere form (roughly 100 μm to 20 mm in diameter) obtained from, for example, Acetobacter xylinum strain ATCC 700718 cultured in a 250 mL flask in with 100 mL of media in an orbital shaking incubator at 175 RPM and 30° C. Cellulose can be aligned cellulose where the cellulose fibers are aligned in an AC electric field including the case where the aligned cellulose are aligned in an electric field during its production in a culture of Acetobacter xylinum. In some cases, the cellulose is freeze-dried and dehydrated where it can be rehydrated in an aqueous solution. This aqueous solution can contain the HA-casein particles.

The cellulose is washed by centrifuge or straining 2-3 times to remove excess particles. It is freeze dried and sealed in a package with a desiccant or in dry air or nitrogen to preserve samples.

In some cases, aqueous solutions containing the HA-casein-cellulose material is further modified by the incorporation of another material to form a gel-like composition. This is accomplished by adding the cellulose, casein, and HA mixture to a mixture of starch, chitosan, or carboxylmethylcellulose in an aqueous solution where the ratio of the content of the starch, chitosan, or carboxylmethylcellulose to total water content is 1:200; 1:100, 2:100, 3:100, 4:100, 5:100, or 10:100.

### Method #3:

κ-casein is added to cellulose in an aqueous solution at a pH of 7-11 and mixed on a rotor for 10-30 minutes at 10 RPM to 50 RPM. The weight ratio of the casein to cellulose is 1:200, 1:100, 1:50, 1:25, 1:10, 1:5 or 1:2. The solid content is 0.5%, 1%, 2%, 3%, 4%, 5%, 10%, or 25%. This cellulose is cellulose derived from plants (including trees) or from bacteria such as Acetobacter xylinum. The cellulose is in the form of a sheet or pellicle obtained from a static bacteria culture (about 0.5 mm to 25 mm thick), a mixture or mass of cellulose obtained from agitated bacteria cultures, in nanowhisker or nanofiber form (fibers measuring 2 nm to 35 nm diameter, 50 nm to 50000 nm length) produced via acid hydrolysis processes, or in pellet or sphere form (roughly 100 μm to 20 mm in diameter) obtained from, for example, Acetobacter xylinum strain ATCC 700718 cultured in a 250 mL flask in with 100 mL of media in an orbital shaking incubator at 175 RPM and 30° C. Cellulose can be aligned cellulose where the cellulose fibers are aligned in a DC or AC electric field including the case where the aligned cellulose are aligned in an electric field during its production in a culture of Acetobacter xylinum. Cellulose is freeze-dried and dehydrated where it would be rehydrated in an aqueous solution. This aqueous solution can contain the casein.

Cellulose is washed by centrifuge or straining 2-3 times to remove excess casein. It is suspended in an aqueous solution where the solid content is 1%, 2%, 3%, 4%, 5%, 10%, 15%, 20%, or 25%. The pH is adjusted to ˜7. Chymosin is added to the solution such that the ratio of chymosin to casein is 1:100000, 1:10000, 1:1000, 1:100, or 1:10 and mixed on a rotor for 10-30 minutes at 10 RPM to 50 RPM. The cellulose is washed by centrifuge or straining 2-3 times to remove excess casein protein fragments. The cellulose is suspended in an aqueous solution where the solid content is 1%, 2%, 3%, 4%, 5%, 10%, 15%, 20%, or 25%. The pH is adjusted to 7-11. HA (particle diameters 2 nm to 2 μm) is added and mixed on a rotor for 10-30 minutes at 10 RPM to 50 RPM. The ratio of the casein and the cellulose mixture to HA is 20:1, 10:1, 5:1, 2:1, 1:1, 1:2, 1:5, 1:10, or 1:20. The cellulose is washed by centrifuge or straining 2-3 times to remove excess HA. It is freeze dried and sealed in a package with a desiccant or in dry air or nitrogen to preserve samples.

In some cases, aqueous solutions containing the HA-casein-cellulose material can be further modified by the incorporation of another material to form a gel-like composition. This is accomplished by adding the cellulose, casein, and HA mixture to a mixture of starch, chitosan, or carboxylmethylcellulose in an aqueous solution where the ratio of the content of the starch, chitosan, or carboxylmethylcellulose to total water content is 1:200; 1:100, 2:100, 3:100, 4:100, 5:100, or 10:100.

### Method #4:

κ-casein is added to HA (particle diameters 2 nm to 2 μm) in an aqueous solution at a pH of ˜7 and mixed on a rotor for 10-30 minutes at 10 RPM to 50 RPM. The ratio of the casein to HA is 1:100, 1:50, 1:25, 1:10, 1:2, 1:1, 2:1, 10:1, 25:1, 50:1, or 100:1. The solid content is 0.5%, 1%, 2%, 3%, 4%, 5%, 10%, or 25%. If desired, the HA is washed to remove excess casein 2-3 times by centrifugation and suspended in an aqueous solution where the solid content is 1%, 2%, 3%, 4%, 5%, 10%, 15%, 20%, or 25%. The pH is adjusted to ˜7. Chymosin is added to the solution such that the ratio of chymosin to casein is 1:100000, 1:10000, 1:1000, 1:100, or 1:10 and mixed on a rotor for 10-30 minutes at 10 RPM to 50 RPM. HA is washed by centrifuge 2-3 times to remove excess casein protein fragments and suspend in an aqueous solution where the solid content is 1%, 2%, 3%, 4%, 5%, 10%, 15%, 20%, or 25%. The pH is adjusted to 7-11. Cellulose is added to the solution. The final ratio of the casein and HA mixture to cellulose is 1:25, 1:10, 1:5, 1:2, 1:1, 2:1, 5:1, 10:1, or 25:1. This cellulose can be cellulose derived from plants (including trees), or from bacteria such as Acetobacter xylinum and can be in the form of a sheet or pellicle obtained from a static bacteria culture (about 0.5 mm to 25 mm thick), a mixture or mass of cellulose obtained from agitated bacteria cultures, in nanowhisker or nanofiber form (fibers measuring 2 nm to 35 nm diameter, 50 nm to 50000 nm length) produced via acid hydrolysis processes, or in pellet or sphere form (roughly 100 μm to 20 mm in diameter) obtained from, for example, Acetobacter xylinum strain ATCC 700718 cultured in a 250 mL flask in with 100 mL of media in an orbital shaking incubator at 175 RPM and 30° C. In some cases, cellulose can be aligned cellulose where the cellulose fibers are aligned in an AC electric field including the case where the aligned cellulose are aligned in an electric field during its production in a culture of Acetobacter xylinum. Cellulose can be freeze-dried and dehydrated where it would be rehydrated in an aqueous solution. This aqueous solution can contain the HA-casein particles. Cellulose is washed by centrifuge or straining 2-3 times to remove excess particles and suspended in an aqueous solution where the solid content is 1%, 2%, 3%, 4%, 5%, 10%, 15%, 20%, or 25%. The pH is adjusted to ˜7. It can be freeze dried and sealed in a package with a desiccant or in dry air or nitrogen to preserve samples.

In some cases, aqueous solutions containing the HA-casein-cellulose material can be further modified by the incorporation of another material to form a gel-like composition. This is accomplished by adding the cellulose, casein, and HA mixture to a mixture of starch, chitosan, or carboxylmethylcellulose in an aqueous solution where the ratio of the content of the starch, chitosan, or carboxylmethylcellulose to total water content is 1:200; 1:100, 2:100, 3:100, 4:100, 5:100, or 10:100.

### Example 2

### Producing Composite Containing Casein Polypeptides, Cellulose, and Polylactic Acid

### Method #1:

Bovine milk casein and cellulose (paper pulp, microfibrillated cellulose, microbial cellulose, or cellulose nanowhiskers) are mixed in weight ratios of 1:200, 1:100, 1:50, 1:25, or 1:10 in water adjusted to a pH of 7-11 using sodium hydroxide. Solid content in the final solution can range from 2% to 95% (e.g., a range of 5% to 25%). The material is mixed and dehydrated using thermal drying or freeze drying. A final mixture is produced containing the dehydrated casein and cellulose mixture, and polylactic acid in dry form (<5% water) in weight ratios of 1:100, 1:50, 1:25, 1:10, 1:5, 1:2, 1:1, 2:1, 5:1, or 10:1. The material is mixed and extruded or molded as is appropriate for a particular application.

### Method #2:

A mixture of bovine milk casein and a calcium containing mineral such as calcium carbonate or calcium phosphate is produced in weight ratios of 1:100, 1:50, 1:25, or 1:10 in water adjusted to a pH of 7-11 using sodium hydroxide. Solid content in the final solution can range from 0.5 to 95% (e.g., a range of 0.5% to 5% 0. The material is mixed and dehydrated using thermal drying or freeze drying. A final mixture containing the dehydrated casein and calcium carbonate, and polylactic acid in dry form (<5% water) is produced in weight ratios of 1:100, 1:50, 1:25, 1:10, 1:5, 1:2, 1:1, 2:1, 5:1, or 10:1. The material is mixed and extruded or molded as is appropriate for a particular application.

### Method #3:

A mixture of bovine milk casein and a calcium containing mineral such as calcium carbonate or calcium phosphate is produced in weight ratios of 1:100, 1:50, 1:25, or 1:10 in water adjusted to a pH of 7-11 using sodium hydroxide. Solid content in the final solution can range from 0.5 to 95% (e.g., a range of 0.5% to 5%). Cellulose (paper pulp, microfibrillated cellulose, microbial cellulose, or cellulose nanowhiskers) is added to a final solid content of 1%, 2%, 3%, 4%, 5%, 10%, 25%, 50%, 75%, 90%, 95%, 98%, or 99%. The material is mixed and dehydrated using thermal drying or freeze drying. A final mixture containing the dehydrated casein, calcium carbonate and cellulose mixture, and polylactic acid in dry form (<5% water) is produced in weight ratios of 1:100, 1:50, 1:25, 1:10, 1:5, 1:2, 1:1, 2:1, 5:1, 10:1, 50:1 or 100:1. The material is mixed and extruded or molded as is appropriate for a particular application.

### Example 3

### Paper, Cardboard, Wood Sheet Coatings

### Method #1:

A mixture of bovine milk casein and a calcium containing mineral such as calcium carbonate or calcium phosphate is produced in weight ratios of 1:100, 1:50, 1:25, 1:10, or 1:5 in water adjusted to a pH of 7-11 using sodium hydroxide. The solid content in the final solution can range from 0.5 to 95% (e.g., a range of 5% to 75%). The composition is applied to paper by using, for example, gravure coating, reverse roll coating, knife over roll (gap) coating, metering rod (Meyer rod) coating, slot die (slot extrusion) coating, immersion (dip) coating, curtain coating, air knife coating, dip roll coating, calendaring process coating, lamination coating, or via spraying as described elsewhere (Coating Technology Handbook; Donatas Satas and Arthur A. Tracton, 2^{nd} edition, 2001, Marcel Dekker, Inc., 270 Madison Ave., New York, N.Y. 10016).

### Method #2:

A mixture of bovine milk casein and a calcium containing mineral such as calcium carbonate or calcium phosphate is produced in weight ratios of 1:100, 1:50, 1:25, 1:10, or 1:5 in water adjusted to a pH of 7-11 using sodium hydroxide. The solid content in the final solution can range from 0.5 to 95% (e.g., a range of 5% to 25%). The material is mixed and dehydrated using thermal drying or freeze drying. A final mixture containing the dehydrated casein and calcium carbonate mixture, and polylactic acid in dry form (<5% water) is produced in weight ratios of 1:50, 1:25, 1:10, 1:5, 1:2, 1:1, 2:1, or 5:1. The composition is applied to paper by using, for example, gravure coating, reverse roll coating, knife over roll (gap) coating, metering rod (Meyer rod) coating, slot die (slot extrusion) coating, immersion (dip) coating, curtain coating, air knife coating, dip roll coating, calendaring process coating, lamination coating, or via spraying as described elsewhere (Coating Technology Handbook; Donatas Satas and Arthur A. Tracton, 2^{nd} edition, 2001, Marcel Dekker, Inc., 270 Madison Ave., New York, N.Y. 10016).

### Method #3:

A mixture of bovine milk casein and a calcium containing mineral such as calcium carbonate or calcium phosphate is produced in weight ratios of 1:100, 1:50, 1:25, 1:10, or 1:5 in water adjusted to a pH of 7-11 using sodium hydroxide. Solid content in the final solution can range from 0.5 to 95% (e.g., a range of 0.5% to 5%). Cellulose (paper pulp, microfibrillated cellulose, microbial cellulose or cellulose nanowhiskers) is added to a final solid content of 5.5%, 6%, 7%, 10%, 15%, 20%, 30%, 40%, 50%, or 75%. The composition is applied to paper by using, for example, gravure coating, reverse roll coating, knife over roll (gap) coating, metering rod (Meyer rod) coating, slot die (slot extrusion) coating, immersion (dip) coating, curtain coating, air knife coating, dip roll coating, calendaring process coating, lamination coating, or via spraying as described elsewhere (Coating Technology Handbook; Donatas Satas and Arthur A. Tracton, 2^{nd} edition, 2001, Marcel Dekker, Inc., 270 Madison Ave., New York, N.Y. 10016).

### Method #4:

A mixture of bovine milk casein and a calcium containing mineral such as calcium carbonate or calcium phosphate is produced in weight ratios of 1:100, 1:50, 1:25, 1:10, or 1:5 in water adjusted to a pH of 7-11 using sodium hydroxide. Solid content in the final solution can range from 0.5 to 95% (e.g., a range of 0.5% to 5%). Cellulose (paper pulp, microfibrillated cellulose, microbial cellulose or cellulose nanowhiskers) is added to a final solid content of 1%, 2%, 3%, 4%, 5%, 6%, 7%, 10%, 15%, or 20%. The material is mixed and dehydrated using thermal drying or freeze drying. A final mixture containing the dehydrated casein, calcium carbonate and cellulose mixture, and polylactic acid in dry form (<5% water) is produced in ratios of 1:50, 1:25, 1:10, 1:5, 1:2, 1:1, 2:1, 5:1, 10:1, 25:1, or 50:1. The composition is applied to paper by using, for example, gravure coating, reverse roll coating, knife over roll (gap) coating, metering rod (Meyer rod) coating, slot die (slot extrusion) coating, immersion (dip) coating, curtain coating, air knife coating, dip roll coating, calendaring process coating, lamination coating, or via spraying as described elsewhere (Coating Technology Handbook; Donatas Satas and Arthur A. Tracton, 2^{nd} edition, 2001, Marcel Dekker, Inc., 270 Madison Ave., New York, N.Y. 10016).

### Example 4

### Aligned Microbial Cellulose for Nerve and Tissue Regeneration

A static culture of Acetobacter xylinum bacteria (for a standard culture media composition, see: Kouda et al., J. Ferment. Bioeng., 83:371-376 (1997)) is started where the culture media is placed in a container containing two plate-like electrodes measuring 1 mm to 10 cm by 1 cm to 10 cm positioned parallel to each other separated by a distance of 0.1 mm, 0.5 mm, 1 mm, 2 mm, 3 mm, 4 mm, 5 mm, 10 mm, 15 mm, 20 mm, or 25 mm. A voltage of 1V, 5V, 10V, 50V, 100V, 250V, 500V, 1000V, or 5000V is applied. An optimal voltage and distance produced a field strength of alignment of 250 V/cm to 5000 V/cm. The frequency of oscillation of the electric field is between 1 kHz and 10 MHz, where an optimal frequency is between 0.1 MHz and 2 MHz. The culture continues for 1-14 days (e.g., 3-7 days). The cells are removed by gently washing in a 10 mM to 1 M solution of sodium hydroxide. The cells are gently washed in deionized water, changing water every 12 hours, for 2 days or until the pH is <7.5. This aligned cellulose is used as a source of cellulose for the applications provided herein (e.g., Examples 1-3).

### Example 5

### Adsorption of Polylactic Acid on Casein Deposited Surface

Poly(lactic acid) (PLA, OLYGOs Bioresin 120, MW: 2500-3500, NatureWorks® LLC), casein proteins (from bovine milk, Sigma), and dimethyl sulfoxide (DMSO, Burdick & Jackson) were used as received. The running buffer and solvent in all experiments were a mixture of 80% phosphate buffered saline (PBS, pH=7.3) and 20% DMSO. The SPR chips (18 mm×18 mm×1 mm) with 10 angstroms of Cr followed by 500 angstroms of Au on standard float glass were purchased from EMF Corporation.

### Surface Plasmon Resonance Imaging

The SPR imaging system (GWC Technologies, SPRimager®) was used to detect the binding between polylactic acid and casein. The SPR imaging system was used as described elsewhere (Brockman et al., J. Am. Chem. Soc., 121:8044-8051 (1999); Jordan et al., Analytical Chem., 69:4939-4947 (1997); and Nelson et al., Anal. Chem., 71:3928-3934 (1999)). Generally, p-polarized collimated polychromatic light was impinged on the prism/gold film/sample assembly at a fixed or changed angles. The p-polarized light elicited the SPR effect. As a result, attenuated light was reflected. Light reflected passed through a narrow band-pass filter and fell upon the CCD camera as a detector. All SPR images were collected using the software V++(Digital Optics).

### Sample Preparation

The SPR chips were soaked in Piranha solution (sulfuric acid: 30% hydrogen peroxide=3:1) for 10-15 minutes. Caseins were dissolved in the running buffer until saturation (˜0.1 mg/mL). 60 mg PLA was dissolved in 4 mL DMSO, and mixed with 16 mL PBS to make a 1 mM solution. The whole experiment was carried out at room temperature.

### Results

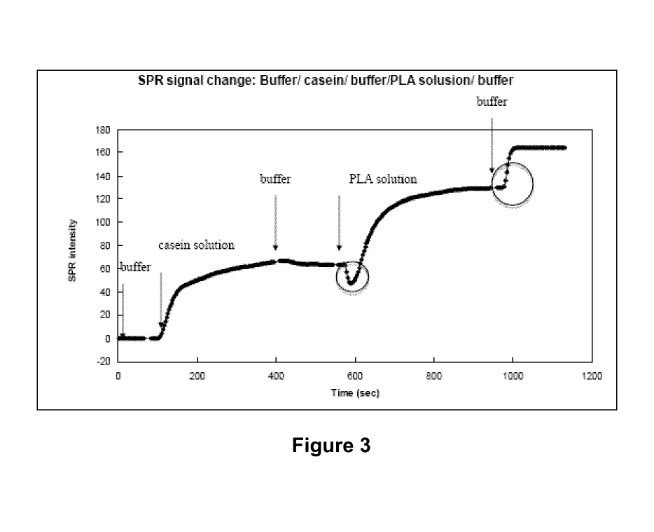

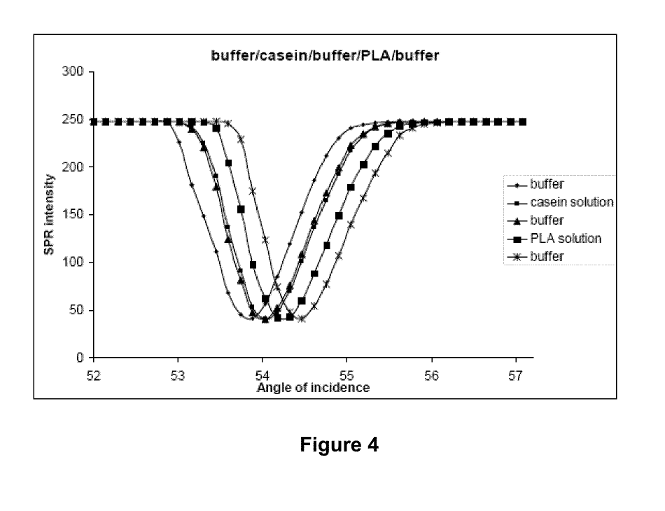

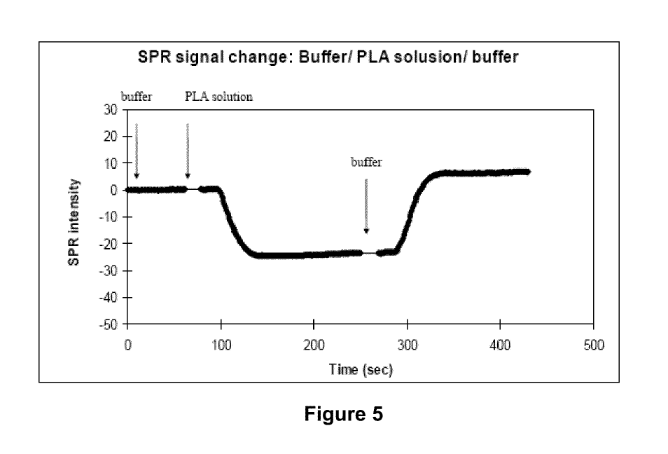

The results for polylactic acid adsorption on casein deposited surface were plotted with the normalized intensity being plotted as a function of time and angle, respectively (FIGS. 3 and 4). The casein was first deposited on the gold chip and bound to the surface (FIG. 3). PLA solution flowed through the casein coated chip and bound to the surface (FIG. 3). The increase in SPR intensity caused by a refractive index change on the surface indicated PLA adsorption. There was a difference of >60 in the normalized SPR intensity indicating binding of the PLA to casein (FIG. 3). The reflectivity changes highlighted by the circles shown in FIG. 3 are present due to the difference in the buffer solution refractive index when PLA is present. This is also shown in FIG. 5.

FIG. 4 reveals the change of SPR angle (the angle of minimum reflected light). A large shift was detected when PLA solution flowed on the casein deposited surface (FIG. 4).

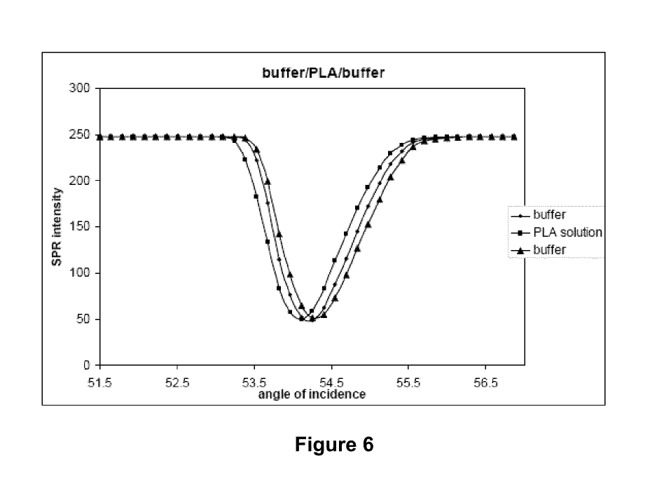

FIGS. 5 and 6 present the results for the control: PLA solution flowed on the bare gold film without casein deposited. The conclusion is that almost no binding between gold surface and polylactic acid was observed as compared to the binding observed in FIGS. 3 and 4, respectively.

There results demonstrate that casein protein can bind to PLA making it a material useful for forming composites with PLA, PLA and cellulose, or PLA, cellulose and calcium containing minerals.

### Example 6

### Templated Growth of Microbial Cellulose for Tissue Regeneration Applications

In some tissue regeneration applications, there can be a need to direct the growth of the cells which form the tissue. An example is the growth of nerve cells. For example, breaks in nerve tissue resulting from injury cannot heal in the event that the separation of the tissue at the break point exceeds a few hundred microns. Such healing would require the organization and pattern of nerve connectivity to be restored which cannot happen naturally when the break exceeds these dimensions. A scaffold material capable of directing nerve tissue growth along the axis of the break could be beneficial to the healing and recovery process.

Such an anisotropic tissue scaffold material is created by using a degradable template and culturing of microbial cellulose. For example, microbial cellulose produced by an organism such as Acetobacter xylinum is cultured in a media containing poly(lactic-co-glycolic acid) or polylactic acid fibers. These fibers can range in diameter from 10 microns to 1 millimeter or 50 microns to 200 microns. Their length can be from 100 microns to 10 millimeters. These fibers are wound around a holder allowing for alignment of the fibers within a bundle. The number of fibers in a bundle can vary so as to control the diameter of the bundle, which can vary from about 500 microns to over 25 millimeters. The holder design is engineered to allow for different bundle shapes, i.e., linear, round, v-shaped, or rectangular shaped. A schematic diagram of a holder with a feature for forming a round-like fiber bundle is shown in FIG. 7. FIG. 8 depicts a schematic illustration of a culturing setup where the holder shown in FIG. 7 is submerged into a culture media growing Acetobacter xylinum and cellulose.

A process for producing the scaffold includes of the following steps: (1) fabrication of a holder for the poly(lactic-co-glycolic acid) fibers as described herein and shown, e.g., in FIG. 7; (2) winding 100 micron diameter fibers around the holder to create a round-like fiber bundle measuring about 5 mm to 10 mm in diameter; (3) mixing a culture media for culturing Acetobacter xylinum; (4) sterilizing the culture media, holder, culture vessel, and poly(lactic-co-glycolic acid) fibers (if needed) by autoclave and/or ultraviolet sterilization processes; (5) filling the culture vessel with media and inserting the holder into the media as shown in FIG. 8; (6) culturing the cellulose for 5-15 days at 26-34° C. allowing the cellulose to grow around and in between the fibers; (7) washing the material in sterile 1 mM NaOH at 80° C. for 2-4 hours to lyse the cells; (8) washing the material gently in a water bath under rocking motion for 4 days or until the pH reaches ˜7.0 while exchanging the water every 12-16 hours to remove the cellular debris, media, and NaOH solution; and (9) if desired, functionalizing the cellulose with polypeptide and/or mineral to form a composite as described herein.

During this process, the poly(lactic-co-glycolic acid) is dissolved leaving behind cylindrical-like holes in the microbial cellulose corresponding to the locations where the poly(lactic-co-glycolic acid) fibers were initially positioned. The diameter of the holes may shrink as the cellulose grows and the poly(lactic-co-glycolic acid) dissolves. This can be used to optimize the hole diameter and amount of microbial cellulose positioned between holes. Nerve or other cells would then grow within these holes and direct the growth of nerve cells along the direction of the holes in the material.

The nano and microscale porosity of the microbial cellulose (about 50 nm to about 2 microns) allows interaction of the axons or dendrites, in the case of nerve tissue, existing in nearby holes but still permit the directed growth of the much larger cells (about 5-20 microns and larger) along the length of the holes along the length of the material. The material is positioned in the area of nerve damage such that the axis of the holes is parallel to the line connecting the ends of the severed nerve tissue.

### Example 7

### Microbial Cellulose Patch for the Delivery of Olfactive Components

The delivery of volatile olfactive components can be substantially enhanced via the use of microbial cellulose. For example, perfume applied to the skin may deliver a detectable aroma for a given period of time depending upon the concentrations of olfactive components and the amount applied to the surface of the skin or clothing without saturating the skin surface or material, which may be undesirable. Microbially produced cellulose can hold an aqueous solution measuring approximately 50 to over 100 times its weight, i.e., 1 mg of microbial cellulose can hold approximately 50 to over 100 mg of water. Microbial cellulose can be formed in the shape of a small patch measuring anywhere from about 2 mm×2 mm×1 mm thick to over 10 cm×10 cm×1 cm thick. A patch containing one or more olfactive components can be loaded into the microbial cellulose via submersion or through lyophilization to dehydrate the material while preserving its unique nanoporosity and mechanical properties and then rehydrating with the solution containing one or more olfactive components. The viscosity of the solution can be tailored through the addition of polysaccharides such as, for example, carboxymethyl cellulose or chitosan. Chitosan can be desirable owing to its cationic nature and binding affinity to cellulose. Increased viscosity of the solution containing one or more olfactive components can provide improved stability to the solution contained in the microbial cellulose patch.

A patch of microbial cellulose containing one or more olfactive components can offer an advantage of providing tailored delivery of a desired aroma over prolonged periods of time owing to its dramatically increased surface area in comparison to a relatively flat surface. Microbial cellulose can be a mesh of nanofibers measuring 10-20 nm in diameter and tens to thousands of microns in length. A uniform delivery of a pleasant aroma over long periods of time is desirable for many applications including body perfume and room fresheners. Microbial cellulose can also be colored to match skin tone, if desired. The microbial cellulose patch can be formed with an adhesive on one side allowing temporary attachment to many surfaces including, for example, skin, wall surfaces, painted surfaces, leather surfaces, vinal surfaces, metal surfaces, wood surfaces, ceramic surfaces, glass surfaces, tile surfaces, Formica surfaces, polished stone surfaces, plastic surfaces, cloth surfaces, cotton surfaces, and polyester surfaces. Such an adhesive can be an adhesive such as one of those described in U.S. Pat. No. 6,177,482.

A particular device could be a microbial cellulose patch where the cellulose is produced from a statically grown culture in the form of a pellicle where the pellicle could be cut to a desired size or shape, which could be circular, square or any other shape. The thickness of the patch can be governed by the culture time, nutrient media composition, and strain of bacteria, which could be, for example, Acetobacter xylinum (e.g., Acetobacter xylinum ATCC #700718). The thickness can range from 0.1 mm to over 10 mm. The bacterial cellulose patch can be lyophilized, fixed to an adhesive located on another carrier substrate such as, for example, a wax paper, and then filled with one or more olfactive components in a solution of a desired viscosity and color. The patches can be placed into a sealed container until use. Use of the patch can involve opening the sealed container, peeling the patch from the carrier substrate, and applying it to the desired surface. One specific example can be a perfume patch that can be temporarily attached to the skin of a person's neck, shoulder, wrist, or other area. Another specific example can be a room freshener patch that can be attached to a wall or inside surface of a car.

### Example 8

### A Coating Containing Cationic and Anionic Polymers, Polypeptides, or Polysaccharides

The following describes the use of oppositely charged polymers for the formation of coatings as described herein, including coatings that can improve the dry strength, wet strength, or liquid barrier properties of a substrate, including substrates composed principally of a polysaccharide such as, for example, cellulose. Two coating methods were developed:

### Method #1

In method #1, the sheet made principally of cellulose pulp is first coated with one or more cationic polymers, polypeptides, or polysaccharides, contained in a liquid solution, to coat the cellulose fibers located principally on the surface of the sheet. The amount of cationic material applied to the surface is at a level that exceeds the amount required to coat the surface fibers leaving some of the cationic material free in the application liquid. The hydrated sheet is allowed to sit for one to 30 minutes or more. Secondly, the cationically coated sheet still hydrated with an application liquid containing the free cations is then coated with one or more anionic polymers, polypeptides, or polysaccharides also contained in a liquid. The sheet is then allowed to sit for one to 30 minutes or more before any supplemental dehydration process is implemented. After this time, the sheet is then pressed and/or heated to dehydrate the sheet. Pressing also increases the density of the sheet.

### Method #2

In method #2, the sheet made principally of cellulose pulp is first coated with one or more cationic polymers, polypeptides, or polysaccharides, contained in a liquid solution, to coat the cellulose fibers located principally on the surface of the sheet. The amount of cationic material applied to the surface is at a level that may or may not exceed the amount required to coat the surface fibers leaving some of the cationic material free in the application liquid. The hydrated sheet is allowed to sit for one to 30 minutes or more. Secondly, the cationically coated sheet still hydrated with an application liquid containing the free cations is then coated with a mixture of one or more anionic and one or more cationic polymers, polypeptides, or polysaccharides also contained in a liquid. The sheet is then allowed to sit for one to 30 minutes or more before any supplemental dehydration process is implemented. After this time, the sheet can then be pressed and/or heated to dehydrate the sheet. Pressing can also increase the density of the sheet. The mixture of one or more anionic and one or more cationic polymers, polypeptides, or polysaccharides is mixed for one to 30 minutes or more to allow higher molecular weight complexes to form in solution, before application to the sheet. The ratio of the total charge contained on the anionic compounds to the total charge contained on the cationic compounds is 1:50; 1:20; 1:10; 1:5; 1:2; 1:1; 2:1; 5:1; 10:1; 20:1; or 50:1. The liquid can be water or a solvent.

### Pulp Solution

0.1% blotting paper solution was made by disintegrating blotting paper (Dick Blick Art Materials) in deionized water at 50 rpm for 2 days.

### Chitosan Solution (CS) Solution

1% CS solution was made by adding 20 g chitosan (50˜190 kDa, from Sigma-Aldrich) to 1960 g of deinonized water first and then adding 20 g of acetic acid (99.7%, EMD Chemical Inc.) drop wise into the mixture while magnetically stirring at 400 rpm. The mixture was stirred for one day and then filtered through 0.7 μm GF/F Whatman filter paper to take out any undissolved chitosan. The filtered solution was sealed and stored in refrigerator. The pH of the 1% chitosan solution was around 4.

### Carboxymethyl Cellulose (CMC) Solution

1% CMC solution was made by adding 240 g CMC (90 kDa, from Sigma-Aldrich) to 1980 g of deinonized water and magnetically stirring at 400 rpm for one day. The mixture was stirred for one day and then filtered through 0.7 μm GF/F Whatman filter paper to take out any undissolved CMC. The filtered solution was sealed and stored in refrigerator. The pH of the 1% CMC solution was around 6.5.

### Mixture (CS+CMC) Solution

Equal amount of 1% CS and CMC solutions were taken and mixed in different dilutions i.e., at 0, 10, and 20 dilutions. The purpose of diluting both the solution before mixing was to avoid the formation of gel, as the presence of gel may create an issue in coating the previously CS coated cellulose sheet uniformly. The particle size of the diluted sample is small which may play a role in binding the particles of the mixture to the previously CS coated sheet.

At 0 and 10 dilution, big and small gels were formed, respectively, whereas at 20 dilution a homogeneous mixture was obtained. Hence, mixture solution made out of 20 times diluted CS and 20 times diluted CMC solutions were used for the second coating material in method #2.

Coating the Sheet with CS and CMC/(CS+CMC)

The hand sheets were made by following the procedure described in TAPPI 205 with some modification to fit the laboratory environment. 1100 mL of the 0.1% paper pulp solution was used to make the pure pulp sheet by using a circular 200 mesh wire having 6 inch diameter. After the sheet was made, it was pressed under 50 psi using a T-Rex system. The hand sheets made were then coated immediately with the above mentioned polymers by following two methods.

### Method #1

The hydrated pure pulp sheet with 6 inch diameter was first sprayed with 1 to 25 mL of 1% CS solution. This was done by spraying the CS solution 1 to 25 times with a laboratory spraying bottle which dispenses approximately 1 mL per single spray. The CS coated sheet was left to dry for one hour, and then 1 to 15 mL of the CMC solution was sprayed on it. The coated pure pulp sheet with CS and CMC was left 24 hours in room temperature to dry.

### Method #2

The hydrated pure pulp sheet with 6 inch diameter was first sprayed with 1 to 25 mL of 1% CS solution. This was done by spraying the CS solution 1 to 25 times with a laboratory spraying bottle which dispenses approximately 1 mL per single spray. The CS coated sheet was left to dry for one hour, and then 1 to 15 mL of the (CS+CMC) mixture solution was sprayed on it. The coated pure pulp sheet with CS and (CS+CMC) mixture was left 24 hours in room temperature to dry.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-20. (canceled)

21. A method for coating a product, wherein said method comprises:
(a) combining casein, a calcium containing mineral, and a biopolymer to form a mixture, and
(b) applying said mixture to a product to form a coated product.

22. The method of claim 21, wherein said calcium containing mineral is calcium carbonate.

23. The method of claim 21, wherein said calcium containing mineral is calcium phosphate.

24. The method of claim 21, wherein said biopolymer comprises polylactic acid.

25. The method of claim 21, wherein said biopolymer comprises poly(lactic-co-glycolic) acid.

26. The method of claim 21, wherein said step (a) comprises mixing said casein and said calcium containing mineral in water.

27. The method of claim 21, wherein said step (a) comprises mixing said casein and said calcium containing mineral in water having a pH of 7-11.

28. The method of claim 21, wherein said step (a) comprises mixing said casein and said calcium containing mineral in solution, wherein the solid content of said solution is from 0.5 percent to 5 percent.

29. The method of claim 21, wherein said step (a) comprises (i) mixing said casein and said calcium containing mineral in solution and (ii) adding said biopolymer to said solution.

30. The method of claim 21, wherein said mixture is applied to said product using a gravure coating process.

31. The method of claim 21, wherein said mixture is applied to said product using an air knife coating process.

32. The method of claim 21, wherein said method comprises mixing said casein and said calcium containing mineral to form a first mixture, dehydrating said first mixture, and adding polylactic acid to said dehydrated first mixture to said mixture.

33. The method of claim 21, wherein said product is a paper pulp product.

34. The method of claim 21, wherein said product is a wood product.

35. A method for creating a composite, wherein said method comprises:
(a) combining casein, a calcium containing mineral, and a biopolymer to form a mixture, and
(b) combining said mixture with a cellulose containing substrate to form a composite.

36. The method of claim 35, wherein said calcium containing mineral is calcium carbonate.

37. The method of claim 35, wherein said calcium containing mineral is calcium phosphate.

38. The method of claim 35, wherein said biopolymer comprises polylactic acid.

39. The method of claim 35, wherein said biopolymer comprises poly(lactic-co-glycolic) acid.
